(19) United States
(12) Patent Application Publication
(10) Pub. No.: US 2021/0236349 A1
(43) Pub. Date: Aug. 5, 2021

(54) ABSORBENT ARTICLES AND ARRAYS OF ABSORBENT ARTICLES COMPRISING DIFFERENT FORMS DESIGNED TO AID IN PHYSIOLOGICAL AND PSYCHOLOGICAL DEVELOPMENT OF INFANTS, TODDLERS, AND CHILDREN

(71) Applicant: The Procter & Gamble Company, Cincinnati, OH (US)

(72) Inventors: Gary Dean LaVON, Liberty Township, OH (US); Masaharu Nishikawa, Cincinnati, OH (US); Keith Richard Willhaus, Cincinnati, OH (US); Nelson Edward Greening, II, Cincinnati, OH (US)

(21) Appl. No.: 17/237,091

(22) Filed: Apr. 22, 2021

Related U.S. Application Data
(63) Continuation of application No. 15/229,794, filed on Aug. 5, 2016.
(60) Provisional application No. 62/201,822, filed on Aug. 6, 2015.

(51) Int. Cl. A61F 13/49; A61F 13/496; A61F 13/491; A61F 13/551

(52) U.S. Cl. CPC A61F 13/49015; A61F 13/55105; A61F 13/491; A61F 13/496

(57) ABSTRACT

The present disclosure is directed to disposable absorbent articles and arrays of disposable absorbent articles which are designed to fit a range of wearers having different physiological, psychological and cognitive needs which can be impacted by product form and level of engagement required during application as well as by the final appearance fit and function of the article.

### CROSS REFERENCE TO RELATED APPLICATIONS

This application is a continuation of U.S. application Ser. No. 15/229,974, filed on Aug. 5, 2016, which claims the benefit, under 35 USC 119(e), to U.S. Provisional Patent Application No. 62/201,822 filed on Aug. 6, 2015, which is herein incorporated by reference in its entirety.

### FIELD

The present disclosure is directed to disposable absorbent articles and arrays of disposable absorbent articles which are designed to fit a range of wearers having different physiological and psychological needs which can be impacted by product form and level of engagement required during application and removal, as well as by the final appearance, fit, and function of the article.

### BACKGROUND

Physiological and cognitive development required for a child to ultimately dress himself or herself involves a variety of skills a child needs to master. These skills include: gross motor skills (that include lifting the arms and legs in specific coordinated motions for example to align with shirt sleeves and pant legs, and balancing themselves to take off shoes and pants), fine motor skills (that involve being able to use fingers to grasp items firmly and also to manipulate small objects (zippers, buttons, buckles, and laces)), and finally cognitive skills (involving the ability to understand and execute a sequence coordinated movements such as putting on clothing, e.g. one leg then the other).

The skills both physiologically and cognitively develop and change significantly by age as described hereinafter. It is believed that it would be beneficial to both the infant and the caregiver to provide absorbent articles that can support and enhance the physiological and cognitive development as the child grows. It is therefore the intent of the disclosure to create an array of articles with specific features that elate with, support and enhance the physiological and cognitive development of the wearer.

The cognitive and physiological development of infants from newborn through the toddler years is well understood. An infant's physical development begins at the head then moves to other parts of the body. For example, sucking comes before sitting, which comes before walking.

Infants, newborn to 2 months, display a number of primitive responses for example the Babinski reflex wherein the toes fan outward when sole of foot is stroked, the Moro reflex (startle reflex), wherein the arms extend arms then retract often triggered by loud sounds or sudden movements and the rooting and sucking reflex wherein the infant turns head in search of nipple when cheek is touched and begins to suck when nipple touches lips. Physically these infants can lift and turn the head when lying on his or her back. Their hands are fisted and the arms are flexed. The neck is unable to support the head when the infant is pulled to a sitting position. Given their lack of stability and mobility and the fact that they are primarily in a prone or reclined position, a taped diaper form is best given the application and removal will be done primarily in the prone position given their inability to sit or stand unaided. In addition, a very soft product would also be beneficial to minimize marking associated with their immobility.

Between 3 and 4 months the infant begins to control hand and feet actions, but these movements are not fine-tuned. The infant may begin to use both hands, working together, to accomplish tasks. The infant is still unable to coordinate the grasp, but swipes at objects to bring them closer. They can also rise up (upper torso, shoulders, and head) with arms when lying face down (on tummy). The infant's neck muscles are developed enough to allow the infant to sit with support, and keep their head up. Most of the primitive reflexes have either already disappeared, or are starting to disappear. Given the transition from primarily laying to some sitting with support, a taped diaper form is best given the application and removal will be done primarily in the prone position. Additional features may include soft materials and structures to minimize marking associated with their level of immobility. In addition, increased flexibility/extensibility, e.g. stretch panels and/or waistbands, may be desired to enable the product to adapt the dimensional changes in body shape associated with the changes in position as the infant goes from laying to sitting.

Infants that are between 5 and 6 months are able to sit alone, without support, for only moments at first, and then for up to 30 seconds or more. The infant can roll from back to stomach. When on tummy, the infant can push up with arms to raise the shoulders and head and look around or reach for objects. Although these infants have advanced physiologically to the point of sitting without support, a taped diaper form is still best given the application and removal will be done primarily in the prone position due to the inability of the wearer to stand. Soft materials and structure will minimize marking associated with the lack of mobility and pressures associated with sitting as the primary position. Increased mobility, rolling, and the variety of positions the infant may assume makes a product having increased flexibility/extensibility, e.g. stretch panels and/or waistbands, desirable to adapt to the dimensional changes in body shape associated with the changes in position as the infant goes from laying to sitting and the increase in mobility.

During the 6 to 9 months age window the infant can begin crawling and can walk while holding an adult's hand. Infants in this age range are also able to sit steadily, without support, for long periods of time and they learn to sit down from a standing position. Infants may also be able to pull into and keep a standing position for a short period of time while holding onto furniture. The increase in mobility, which now includes standing with help, requires a significant of flexibility/extensibility. A taped diaper would be the best article form given the inability to stand unaided. Softness is needed to help minimize marking associated with immobility, albeit less than before, and extensibility provides an adequate level of adaptation of the waist to compensate for the dimensional changes of the anatomy associated with positional changes, laying to sitting and rolling front to back. Providing a structure with sufficient flexibility/extensibility is key to allow the infant to develop physiologically as they learn to sit, roll and crawl.

Infants between 9 and 12 months begin to crawl on all fours and can attain a sitting position unaided. These infants also begin to balance while standing alone with support for short periods at a time. They can also take small steps while holding an adults hand; may take few steps alone. The increase in mobility, which now includes standing alone with support or with help, requires a significant level of flexibility/extensibility as enabled by features like stretch panels and/or waistbands. A taped diaper or a bridging product, e.g. a refastenable pant article, are the best articles for this stage given the ability to stand with support but inability to stand unaided and inability to balance without support or help. Both forms enable application in a prone position while the bridging product, refastenable pant, enables application to a standing child by a caregiver even if the child requires support to stand. Providing products with sufficient flexibility/extensibility are key to allow the infant to develop physiologically as they learn to sit, crawl and stand.

The 12 to 18 month age range sees a significant increase in mobility as the child learns to walk alone unstably at first and more stably over time. These children can stoop from a standing position and return to a standing position. These children also begin to climb up on to furniture. They can also walk or climb up stairs with help. The 12 to 18 month development window also sees the emergence of self help skills. In the 12 to 15 month window the infant can feed self with fingers and can remove certain items of clothing, e.g. hat, shoes and socks. During the 15 to 18 month window they can chew most foods well. They can open and close doors as well as hold a cup and drink. They also begin to imitate housework and are able to identify and retrieve familiar objects upon request. The increase in mobility, which now includes standing alone and walking, albeit unstably at first requires an article having and increased level of flexibility/extensibility. A taped diaper or a bridging product, e.g. a refastenable pant article, are the best articles for this stage given the ability to stand unaided but lack of balance at the beginning of the range. Both forms enable application in a prone position while the bridging product, refastenable pant, enables application to a standing child even if the child needs some level of support to stand stably. Providing product structures with sufficient flexibility/extensibility is key to allow the infant to develop physiologically as they learn to sit, crawl, stand and start to walk. The ability to choose between laying changes standing changes based on the physiological and cognitive development of the child allows the caregiver to match the change process with their level of development and to further enhance their overall development process. A bridging product enables both types of changes in a single product form.

During the 18 to 24 month timeframe infants develop the ability to run, albeit stiffly. They can also push and pull large objects as well as carry larger objects and maintain balance. They can also come down stairs on abdomen or bottom as well as seat self in small chair. Self-help skills at this age include helping dress and undress self as well as asking for food and drink through vocalizing and gesturing. They also begin to use a spoon, indicate when they may have a wet or soiled diaper, pull person to show them something. The increase in mobility, which now includes standing alone stably and walking requires a significant level of flexibility/extensibility. A bridging product, e.g. a refastenable pant article, provides the best article for this stage given the improvement in the ability to stand. An underwear-like non-refastenable pant may be leveraged at the end of this stage depending on physiological development, primarily balance and/or cognitive development, ability to execute sequential movements necessary for application and/or emotional need, self esteem. The bridging article enables application in a prone position as well as a standing position, which may be needed for the younger children in this age range. The underwear like article enables application to a standing child and requires the child to be able to stand stably and execute a series of sequential movements. The caregiver may choose either a bridging product or underwear like pant product based on their child's level of physiological development and/or cognitive development and/or emotional needs which means their needs are being addressed at a foundational level.

The 24 to 30 month timeframe sees continued mobility development. These infants can jump in place, walk on tiptoes, walk up and down steps (both feet on each step), walk backward and run headlong. The list of self-help skills in the 24-30 month timeframe include pulling on socks, pulling on pants and shorts, drinking from a cup without spilling, helping put things away and beginning toilet training. By 30-36 months these infants are actively toilet training, dressing with supervision and can balance on one foot for 5 seconds or longer. At this stage a significant increase in self-help is recognized as well as increases in mobility, which now includes standing alone, walking, running, climbing, etc. These changes as well as the self-help desire to dress and undress themselves requires a significant level of flexibility/extensibility, as provided by elastomeric belt flaps, to enable ease of application/removal. An underwear-like non-refastenable pant is the best article for this stage given its more underwear like appearance, which serves the emotional needs and the underwear application style, which enhances the cognitive skills and helps with the physiological development. The underwear like article enables application to a standing child as well as self-application, which further enhances the physiological, cognitive and emotional development of the wearer. The underwear-like pant provides the most underwear experience, more like an adult.

In many markets as infants get older the caregivers want to transition them from an open form product like a taped diaper to a closed form product like a pant that has a more underwear-like appearance. This is often an abrupt change for the wearer and as such many wearers are not adequately prepared physiologically, cognitively or emotionally. Although a pant form has an underwear-like appearance and can play a role in the child's psychological (emotional) and physiological (cognitive) development. The transition within a given array of products of the prior art is far too abrupt. The physiological/cognitive development needed to enable the coordinated movements required to apply and remove a pant style product may be such that a transition from an open form, taped diaper, to a closed form pant product in one step presents an increased challenge and as such can introduce increased stresses and disappointments.

Therefore, having an open form, taped diaper, a closed form pant and a bridging product, such as a pant with fasteners to allow application as a taped diaper or as an underwear-like pant enables development, physiologically and cognitively, on a continuum that mirrors the physiological and cognitive development of the wearer thereby providing a smooth transition from one form to the other that is more commensurate with the level of physiological and psychological development of the child and more beneficial from a developmental standpoint. The end point of the continuum of products is a more underwear-like product which allows the child to experience application and removal similar to adult underwear and as such helps them develop physiologically by developing a different range of and quality of motion and cognitively, coordination, balance and ability to plan ahead and carry out a sequence of movements, etc. In addition, the form provides an emotional attraction for them and helps with their self-esteem as they are moving to “big boy” and “big girl” pants thereby positively impacting their psychological needs.

It has been discovered that pant products comprising elastic strands in the waist and sides of the pant the greatest level of extensibility and therefore provide the most underwear-like application and removal, as well as underwear-like appearance and therefore play a significant role in physiological, cognitive and emotional development. Taped products on the other hand do not provide these development opportunities, as they require the infant or toddler to be laid on their back and the product applied on them while they are prone. Several discrete flap pant products (e.g., a center chassis with 2 or 4 discrete side flaps or panels) exist in the market, refastenable discrete flap pants (pants with refastenable seams—often at the sides of the product) and non-refastenable discrete flap pants (pants with permanent seams—often at the sides of the product). Both pant types come to the consumer packaged in a closed form with a preformed waist opening and pair of leg openings. Some caregivers desire the refastenable pants because they provide the ability to apply them as a pant and as a taped diaper since the seam can be opened and reclosed. Although applying like a taped diaper is deficient from a development standpoint it does provide a significant convenience benefit to caregivers especially when away from home since they do not need to remove the shoes or pants to apply the refastenable discrete flap pant. This level of application flexibility also provides a continuum, bridging product, in application from a taped diaper, open form, product to a closed form, underwear-like pant product. Underwear-like belt flap pants (e.g., a center chassis with 1 or 2 continuous belts) in the market today currently do not offer this flexibility in application. However, belt flap pants do provide the greatest opportunity for development, physiologically and psychologically, cognitively and emotionally.

It is therefore the intention of this disclosure to provide unique arrays of open and closed form articles. It is further an object of the present disclosure to provide unique arrays that comprise different pant forms, including providing (1) a pant comprising discrete side panels and (2) a pant comprising belts. Still further, it is an object of this disclosure to provide arrays comprising belted pants comprising a plurality of belt strands. A further object of the present disclosure is to provide different pant forms comprising new combinations of refastenable and permanent side seams.

It is also an object of the present disclosure to provide an array of taped and pant products that meet the needs of the child early in their development, as well as providing a selection of pant products that includes a range of taped and pant products that cover the spectrum of need—including (1) a discrete flap pant product that can be applied as a discrete refastenable flap pant or as a taped diaper, for maximum convenience to the caregiver, and as a transition between taped diapers and (2) a belt flap pant product that offers the most underwear-like structure thereby providing a continuum of offerings to meet the changing needs of the child thereby maximizing the child's development.

It has been discovered that there are a number of product structural features that are important to meet the needs of the various users. It is important that the waist circumference of the product itself closely matches and conforms to the shape of the body during use. This conformity is easily achieved by open form products (e.g., taped diapers or open refastenable pants), because the caregiver applies the product to the wearer adjusting each individual product to fit properly at the waist. It is much more difficult to have a closed form pant product fit well at the waist and still be easy to apply in the closed form given the need to pull it up over the buttocks and hips to the waist. Closed form discrete flap pants have typically been made with elastomeric films and as such the side flaps often have higher modulus than the belted portion of the belt flap pants which typically comprise different stretch materials like elastomeric strands.

A refastenable discrete flap pant can help with this contradiction of higher modulus and need to pull-on especially when applied like an open form product, taped diaper. Underwear-like pants, especially those comprising a flap belt construction with strands have a greater degree of extensibility in the waist and so are easier to apply while providing the proper contraction force for fit at the waist once applied. The result is a product providing fit that is more tailored to the body shape and that provides more effective gasketing, while being easy for the caregiver and/or wearer to apply and remove like underwear.

It is therefore the intention of the present disclosure to (1) create an array of products that meets multiple needs of the wearer and the caregiver by providing a first product in an open form for use on infants who are small and/or lack coordination or muscle tone and/or cognitive development, to (2) enable proper application of a closed form article, a second product in the array that can act as a bridging product being provided in a closed form with the ability to apply the product in a closed form, to encourage or enable development or as an open form, enabled by a refastenable feature provided on the pant, and to (3) offer a third product in a closed form that is designed specifically for application in a closed form. The array of products is specifically designed to help encourage and enable physiological and psychological development of the wearer.

An additional intention of the present disclosure is to provide a discrete underwear-like product for control of urinary incontinence beyond the typical potty training stage—for example for children with nocturnal enuresis. In such cases it may be beneficial to provide in addition to the products described above, a durable or semi-durable chassis, underwear, that can be worn with or without an absorbent insert such that a wearer can use it like normal underwear and then insert the absorbent at night to help contain and control urine discharges associated with certain medical conditions like nocturnal enuresis. For this stage of wearer the product may desirably be in a closed pant form to provide an underwear-like appearance and because the wearer has developed to a more mature point than a pre-potty trained child with regard to physiological and cognitive development.

These are all objects of the present disclosure. Embodiments of the present disclosure may combine various objects mentioned. A particular embodiment may, but need not, embody every object as described.

### SUMMARY OF THE DISCLOSURE

In one embodiment, an array may comprise 3 or more different absorbent article forms and may comprise three or more different sizes of the different disposable absorbent article forms. The array may comprise first, second and/or third packages comprising first, second and/or third disposable absorbent articles. Each of the first, second and/or third absorbent articles may comprise a chassis having a topsheet, backsheet and an absorbent core. The first absorbent article may be a first size and may be in an open form, taped diaper. The second absorbent article may be a second size and may be in a closed form with a refastenable seam, enabling application in either a closed form, as a pant, or an open form, as a taped diaper. The third absorbent article may be a third size and may be in a closed form enabling application as a pant, underwear. The first, second and/or third packages may comprise products intended for substantially the same size wearer and thus have substantially the same size range since weight and shape can vary for a given age and/or level of cognitive, psychological and physiological development. The first, second and/or third packages may also comprise the same brand name and/or sub-brand name and/or the same trademark registration and/or may have been manufactured by or for a common manufacturer. The array may be available at a common point of sale, in the same shelf display within a retail establishment or as an array available on-line at a common point of sale.

### BRIEF DESCRIPTION OF THE DRAWINGS

FIG. 1 is a side view of a side panel pant with refastenable side seams

FIG. 2 is a plan view of a side panel pant with refastenable seams

FIG. 3 is a cross section view of the pant of FIG. 1 at the refastenable seam.

FIG. 4 is a front view of a side panel pant on a wearer comprising graphics disposed on a front of a chassis and graphics disposed on multiple side panels, including front and back side panels.

FIG. 5 is a back view of the pant of FIG. 4.

FIG. 6 is a perspective view of a side panel pant with permanent side seams.

FIG. 7 is a plan view of the side panel pant of FIG. 6.

FIG. 8 is a front view of a belt flap pant on a wearer comprising graphics disposed on a belt flap and chassis.

FIG. 9 is a back view of the belt flap pant of FIG. 8.

FIG. 10 is a perspective front view of a belt flap pant comprising discrete belt flaps.

FIG. 11 is a plan view of the belt flap pant of FIG. 10.

FIG. 12 is a cross section view of the belt flap pant of FIG. 11 taken along the longitudinal axis 42, showing the longitudinally opposing discrete belt flaps.

FIG. 13 is a cross section view of an alternate embodiment of the belt flap pant of FIG. 11 taken along the longitudinal axis 42, showing longitudinally opposing discrete belt flaps.

FIG. 14 is a cross section view of an alternate embodiment of the belt flap pant of FIG. 11 taken along the longitudinal axis 42, showing longitudinally opposing discrete belt flaps.

FIG. 15 is a perspective front view of a belt flap pant comprising graphics disposed on a belt flap and chassis.

FIG. 16 is a perspective front view of a belt flap pant comprising graphics disposed on a belt flap along a waist edge and along a leg edge of the belt flap.

FIG. 17 is a plan view of a belt flap pant wherein opposing belt flaps share a common belt flap layer.

FIG. 18 is a cross section view of the pant of FIG. 17 taken along the longitudinal center line 42, showing the opposing belt flaps comprising a common inner belt layer and a common outer belt layer.

FIG. 19 is a cross section view of an alternate embodiment of the belt flap pant of FIG. 17 taken along the longitudinal axis 42, showing the opposing belt flaps comprising a common outer belt layer.

FIG. 20 is a cross section view of an alternate embodiment of the belt flap pant of FIG. 11 taken along the longitudinal axis 42, showing the opposing belt flaps comprising a common inner belt layer.

FIG. 21 is a front view of a belt flap pant on a wearer comprising graphics disposed on the belt flap and chassis.

FIG. 22 is a perspective front view of a belt flap pant comprising a refastenable side seam.

FIG. 23 is a plan view of a belt flap pant of FIG. 22.

FIG. 24 is a cross section of belt flap pants being made, showing a configuration of a refastenable seams of a first pant and a second pant.

FIG. 25 is a cross section of belt flap pants being made, showing a configuration of a refastenable seams of a first pant and a second pant.

FIG. 26 is a cross section of belt flap pants being made, showing a configuration of a refastenable seams of a first pant and a second pant.

FIG. 27 is a cross section of belt flap pants being made, showing a configuration of a refastenable seams of a first pant and a second pant.

FIG. 28 is a cross section of belt flap pants being made, showing a configuration of a refastenable seams of a first pant and a second pant.

FIG. 29 is a cross section of belt flap pants being made, showing a configuration of a refastenable seams of a first pant and a second pant.

FIG. 30 is a perspective view of a belt flap pant comprising belt flaps of differing longitudinal length.

FIG. 31 is a plan view of the belt flap pant of FIG. 30.

FIG. 32 is a perspective side view of a belt flap pant showing differing longitudinal lengths of a front belt and a back belt at a side seam.

FIG. 33 is a perspective front view of a belt flap pant comprising a shaped, non-rectangular belt flap.

FIG. 34 is a plan view of a continuous web of belt flap pants comprising shaped front and back belt flaps.

FIG. 35 is a perspective side view of a belt flap pant comprising a shaped, non-rectangular belt flap.

FIG. 36 is a plan view of a belt flap pant comprising a shaped, non-rectangular back belt flap.

FIG. 37 is a perspective side view of the belt flap pant of FIG. 36.

FIG. 38 is a plan view of a taped diaper comprising a rectangular chassis, a pair of shaped discrete elastomeric side flaps and a pair of shaped discrete ears.

FIG. 39 is a plan view of a taped diaper comprising a pair of discrete elastomeric side flaps and shaped chassis with integral ears.

FIG. 40 is a plan view of a taped diaper comprising a pair of non-elastomeric side flaps and a waistband disposed between the side flaps.

FIG. 41 is a plan view of a taped diaper comprising a shaped, non-rectangular, chassis and waistband disposed between a pair of fasteners.

FIG. 42 is a perspective view of a taped diaper comprising a belt flap disposed in one waist region and fastened to the opposing waist region.

FIG. 43 is a plan view of a taped diaper comprising a shaped, non-rectangular, belt flap in one waist region.

FIG. 44 is a plan view of a taped diaper comprising a pair of discrete elastomeric side flaps and a rectangular chassis.

FIG. 45 is a plan view of a taped diaper comprising a pair of discrete shaped side flaps, a pair of discrete ears and a rectangular chassis.

FIG. 46 is a plan view of a taped diaper comprising a pair of discrete side flaps and a pair of ears and a shaped chassis.

FIG. 47 is a plan view of a taped diaper comprising a pair of integral elastomeric side flaps and a pair of integral ears and a shaped chassis.

FIG. 48 is a perspective front view of a durable/semi-durable pant style absorbent article and a disposable insert.

FIG. 49 is a cross section view of a durable/semi-durable pant style absorbent article and a disposable insert.

FIG. 50 is a plan view of a durable/semi-durable taped diaper style absorbent article and a disposable insert.

FIG. 51 is a perspective view of a durable/semi-durable taped diaper style absorbent article and a disposable insert.

FIG. 52 is a cross section view of an inner leg cuff and outer leg cuff structure formed by a folded nonwoven web.

FIG. 53 is a cross section view of a pair of opposing inner leg cuff and outer leg cuff structures disposed on a topsheet layer.

FIG. 54 is a cross section view of an absorbent article comprising a pair of opposing inner leg cuff and outer leg cuff structures.

FIG. 55 is a plan view of a belt flap pant comprising an absorbent core comprising longitudinally extending core channels.

FIG. 56 is a plan view of a belt flap pant comprising an absorbent core comprising with laterally extending core channels and longitudinally extending core channels.

FIG. 57 is a cross section view of the absorbent core comprising an acquisition layer and a storage layer comprising core channels.

### DETAILED DESCRIPTION

### Definitions

As used herein, the term “absorbent article” refers to taped diapers, open form, and pull-on garments, closed form, with or without refastenable seams intended to be worn by incontinent individuals to absorb and contain urine and/or feces.

“Pull-on garment” or “pant” means articles of wear which as packaged have a defined waist opening and a pair of leg openings and which are intended to be pulled onto the body of the wearer by inserting the legs into the leg openings and pulling the article up over the waist.

“Disposable” means garments, which are not intended to be laundered or otherwise restored or reused as a garment (i.e., they are intended to be discarded after a single use and to be recycled, composted or otherwise disposed of in an environmentally compatible manner). The pull-on garment may be “absorbent” such that it absorbs and contains the various exudates discharged from the body.

“Closed form” means opposing waist regions are joined, as packaged, either permanently or refastenably to form a continuous waist opening and leg openings.

“Open form” means opposing waist regions are not initially joined to form a continuous waist opening and leg openings but comprise a closure means such as a fastening system to join the waist regions to form the waist and leg openings before or during application to a wearer of the article.

“Array” means a display of packages comprising disposable absorbent articles of different article constructions (e.g., different elastomeric materials [compositionally and/or structurally] in the side panels, side flaps and/or belts flaps, different graphic elements, different product structures, fasteners or lack thereof) said packages having the same brand and/or sub-brand and/or the same trademark registration and/or having been manufactured by or for a common manufacturer and said packages available at a common point of sale, e.g. oriented in proximity to each other in a given area of a retail store. An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up.

“On-line Array” means an “Array” distributed by a common on-line source.

As used herein, the terms “elastic,” “elastomer,” and “elastomeric” refer to a material which generally is able to extend to a strain of at least 50% without breaking or rupturing, and is able to recover substantially to its original dimensions, accounting for set, after the deforming force has been removed.

The term “channel”, as used herein, is a region or zone in an absorbent material layer that has a substantially lower basis weight (e.g., less than 50%, less than 70%, less than 90%) than the surrounding material in the material layer. The channel may be a region in a material layer that is substantially absorbent material-free (e.g., 90% material-free, 95% material-free, or 99% material-free, or completely material-free). A channel may extend through one or more absorbent material layers. The channel generally has a lower bending modulus than the surrounding regions of the absorbent material layer, enabling the material layer to bend more easily and/or rapidly distribute more bodily exudates within the channel than in the surrounding areas of the absorbent material layer. Thus, a channel is not merely an indentation in the material layer that does not create a reduced basis weight in the material layer in the area of the channel.

### Absorbent Articles

The arrays of the present disclosure may comprise absorbent articles 100 of differing structure and/or form that are generally designed and configured to manage bodily exudates such as urine and/or feces.

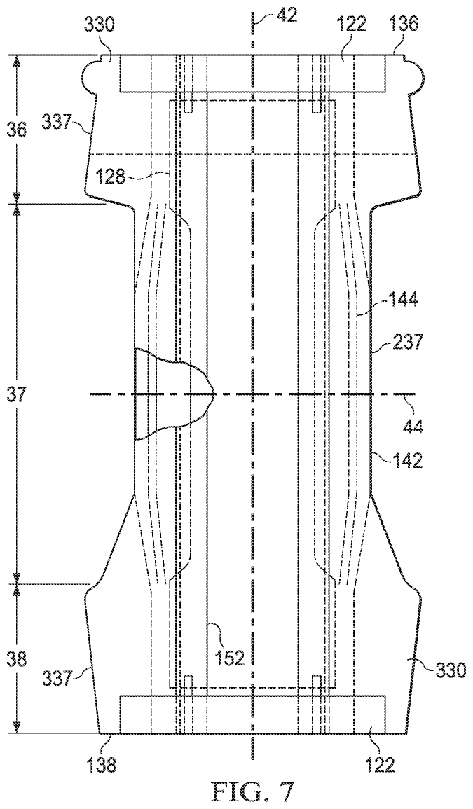

In one embodiment, the absorbent articles 100 of the array may comprise a chassis 200 comprising a topsheet 124, a backsheet 125, and an absorbent core 128 disposed at least partially between the topsheet 124 and the backsheet 125. The chassis 200 may further comprise an inner leg cuff 150 and an outer leg cuff 140. Various chassis embodiments are shown in FIGS. 7, 39, 41 and 54.

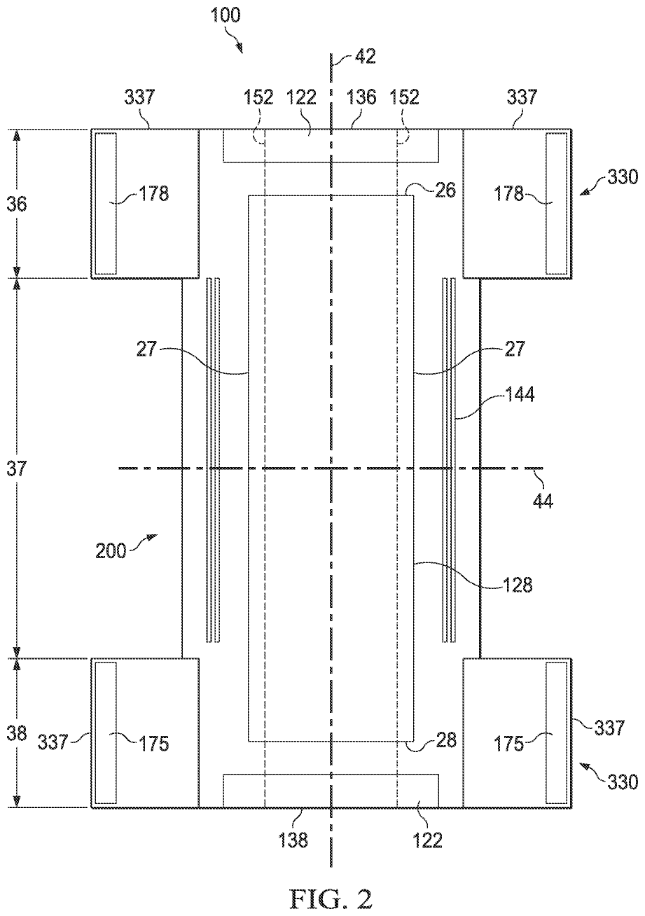

In one embodiment, referring to FIG. 2, one end portion of an absorbent article 100 of the array may be configured as a front waist region 36 and the longitudinally opposing end portion may be configured as a back waist region 38. An intermediate portion of the absorbent article 100 extending longitudinally between the front waist region 36 and the back waist region 38 may be configured as a crotch region 37. In one embodiment, although not illustrated as such, the length of each of the front waist region 36, the back waist region 38 and the crotch region 37 may be about ⅓ of the length of the absorbent article 100, for example. In other embodiments, the length of each of the front waist region 36, the back waist region 38, and the crotch region 37 may have other dimensions. In various embodiments, the absorbent article 100 may have a laterally extending front waist end edge 136 in the front waist region 36 and a longitudinally opposing and laterally extending back waist end edge 138 in the back waist region 38.

In one embodiment, referring to FIG. 2, a chassis 200 of the absorbent article 100 may comprise a first longitudinally extending side edge 237a and a laterally opposing and second longitudinally extending side edge 237b. Both of the side edges 237 may extend longitudinally between the front waist end edge 136 and the back waist end edge 138. The chassis 200 may form a portion of the laterally extending front waist end edge 136 in the front waist region 36 and a portion of the longitudinally opposing and laterally extending back waist end edge 138 in the back waist region 38. Furthermore, the chassis 200 may comprise an chassis interior surface 202, an chassis exterior surface 204, a longitudinal axis 42, and a lateral axis 44. The longitudinal axis 42 may extend through a midpoint of the front waist end edge 136 and through a midpoint of the back waist end edge 138, while the lateral axis 44 may extend through a midpoint of the first side edge 237a and through a midpoint of the second side edge 237b.

In an alternative embodiment, referring to FIG. 11, a chassis 200 of the absorbent article 100 may comprise a first longitudinally extending side edge 237a and a laterally opposing and second longitudinally extending side edge 237b. The chassis 200 may have a length measured along the longitudinal axis 42 that is less than the length of the absorbent article 100. Both of the side edges 237 of the chassis may not extend longitudinally to one or both of the front waist end edge 136 and the back waist end edge 138. The chassis 200 may not form a portion of one or both of the laterally extending front waist end edge 136 in the front waist region 36 and the longitudinally opposing and laterally extending back waist end edge 138 in the back waist region 38. Furthermore, the chassis 200 may comprise an chassis interior surface 202, an chassis exterior surface 204, a longitudinal axis 42, and a lateral axis 44. The longitudinal axis 42 may extend through a midpoint of the front waist end edge of the chassis and through a midpoint of the back waist end edge of the chassis, while the lateral axis 44 may extend through a midpoint of the first side edge 237a and through a midpoint of the second side edge 237b.

In various embodiments, a portion of or the whole absorbent article 100 may be made to be laterally extensible. The extensibility of the absorbent article 100 may be desirable in order to allow the absorbent article 100 to conform to a body of a wearer during movement by the wearer. The extensibility may also be desirable, for example, in order to allow the caregiver to extend the front waist region 36, the back waist region 38, the crotch region 37, and/or the chassis 200 to provide additional body coverage for wearers of differing size, i.e., to tailor the fit of the absorbent article 100 to the individual wearer and to aide in ease of application. Such extension may provide the absorbent article 100 with a generally hourglass shape, so long as the crotch region 37 is extended to a relatively lesser degree than the waist regions 36 and/or 38. This extension may also impart a tailored appearance to the absorbent article 100 during use.

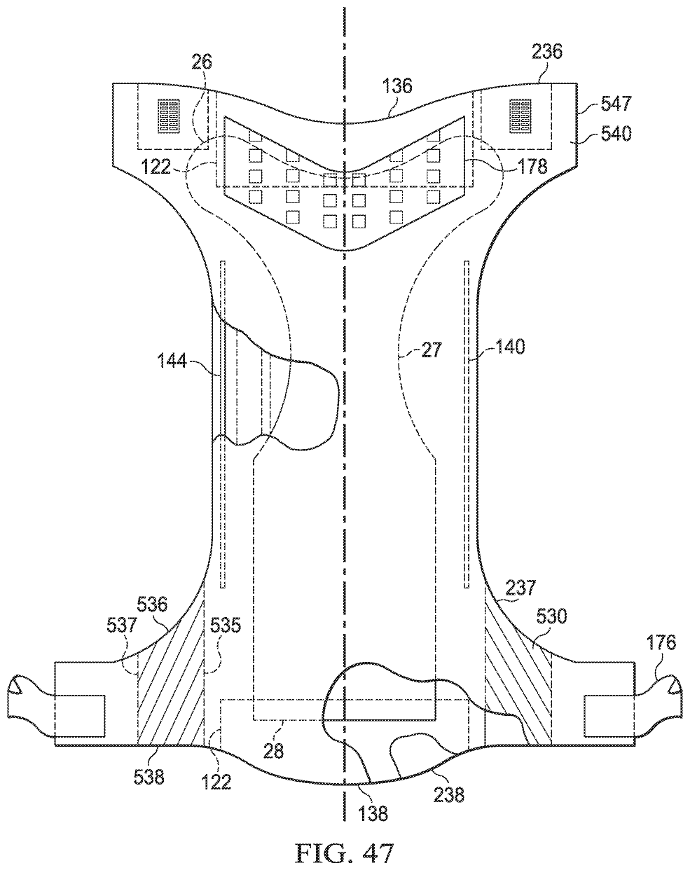

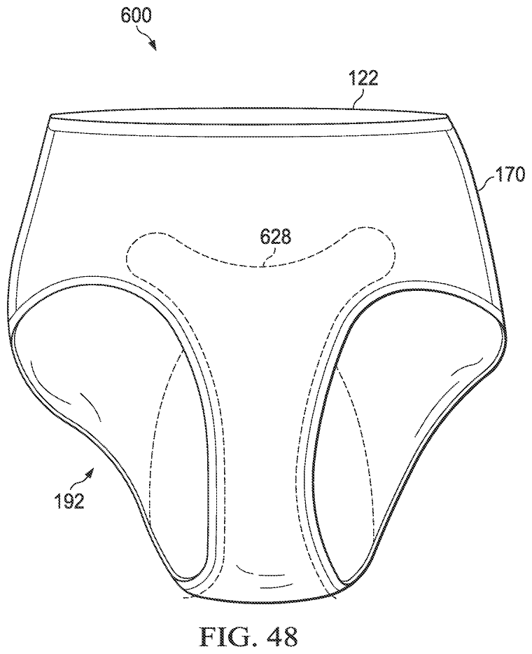

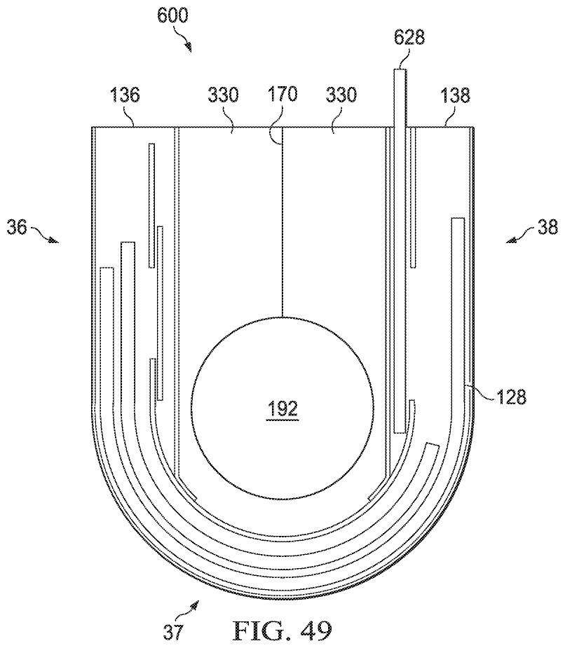

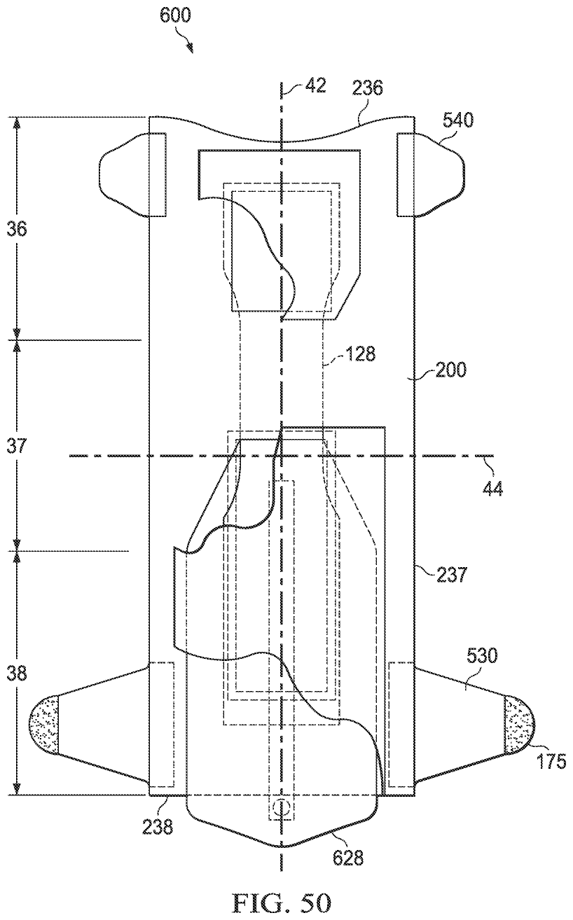

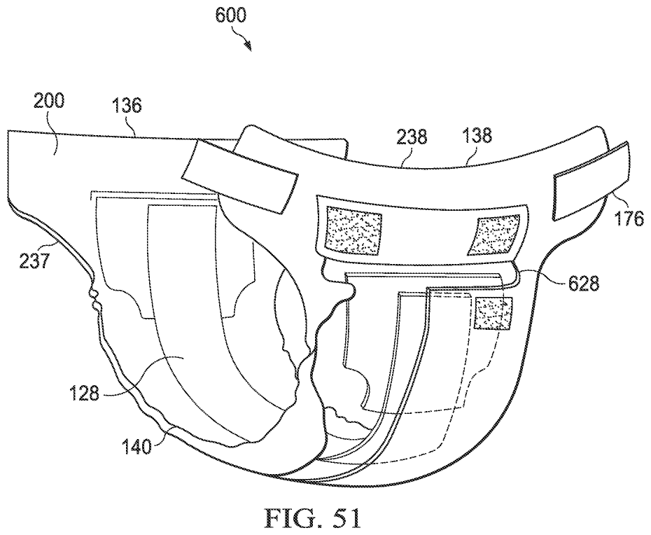

The chassis 200 may be substantially rectangular and may have discrete side panels 330, side flaps 530 and/or ears 540 joined to the chassis 200 at or adjacent the chassis side edges 237 in one or both of the front waist region 36 and back waist region 38. Portions of one or more of the chassis side edges 237, the chassis front end edge 236 and the chassis back end edge 238 may be arcuate or curved either convexly or concavely one embodiment is shown in FIG. 47. For embodiments wherein the chassis 200 is non-rectangular, shaped, the chassis 200 may comprise integral side panels 330, integral side flaps 530 or integral ears 540 formed by one or more of the outer cover nonwoven, backsheet film, outer leg cuff material, topsheet or core wrap disposed in one or both of the front and back waist regions. In certain embodiments the chassis may be shaped or non-rectangular, in one waist region and substantially rectangular in the opposing waist region. Alternatively the chassis may be substantially rectangular in one or both of the waist regions and non-rectangular in the crotch region.

Any or all portions of the absorbent article 100 may comprise a bacteriophage composition as described in U.S. Ser. No. 61/931,229, titled DISPOSABLE ABSORBENT ARTICLES COMPRISING BACTERIOPHAGES AND RELATED METHODS, and filed on Jan. 24, 2014.

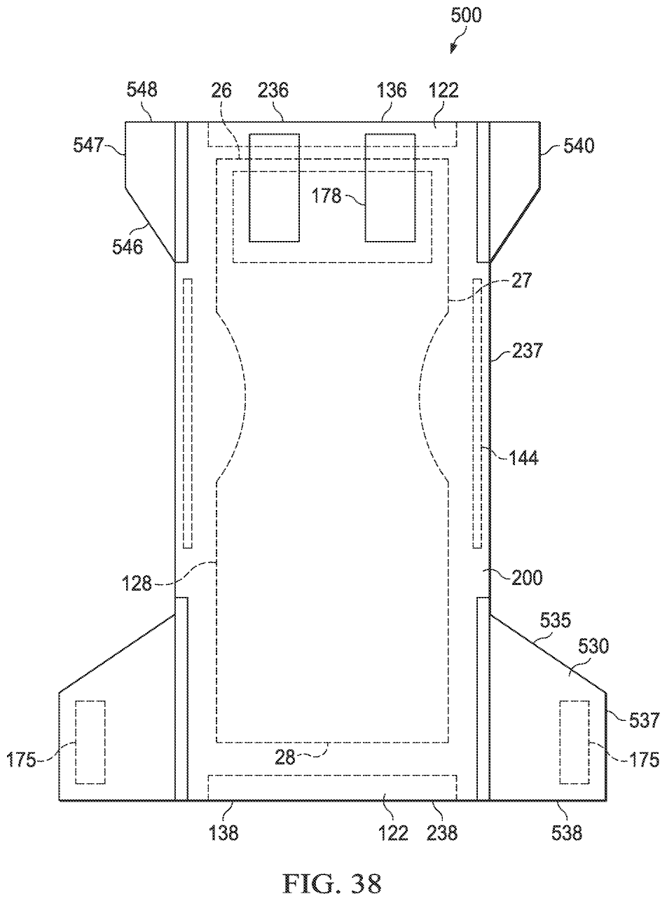

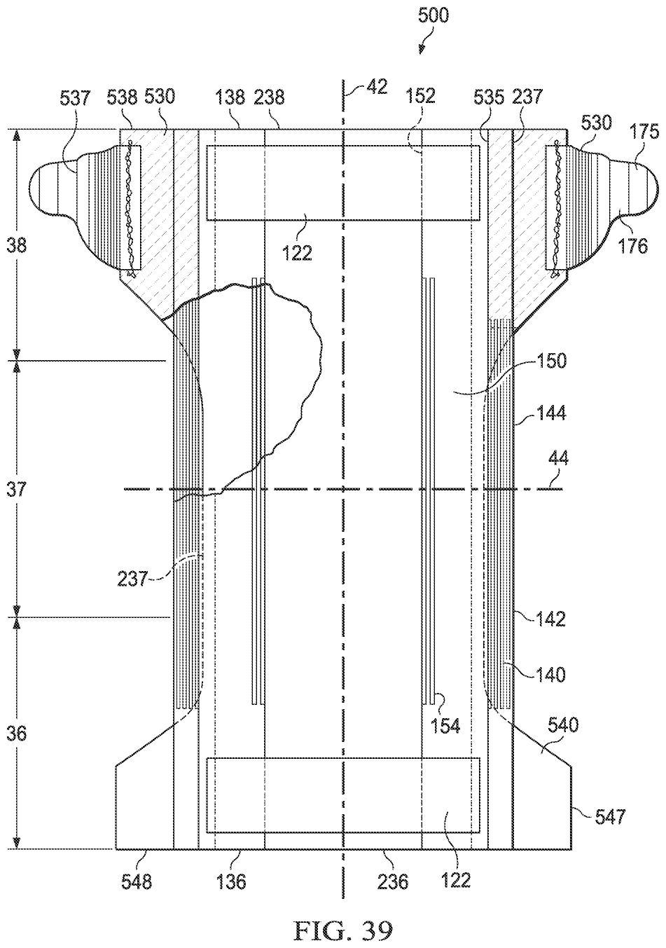

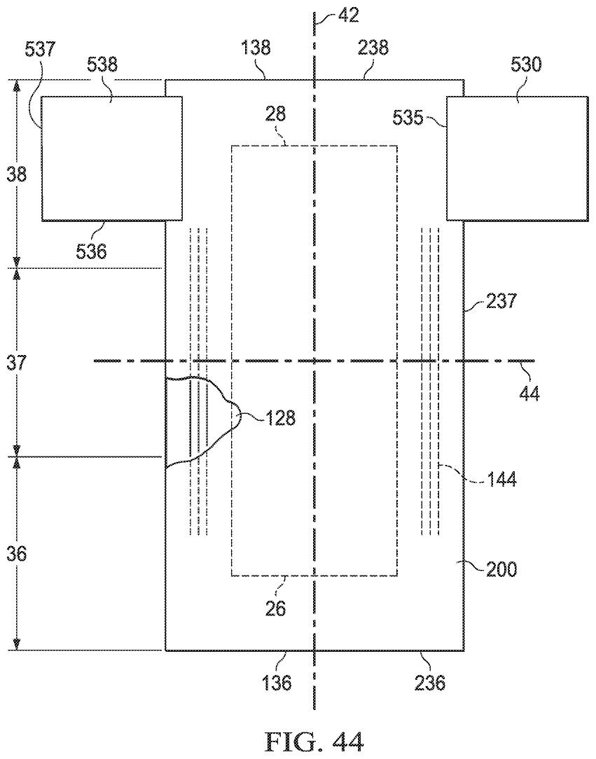

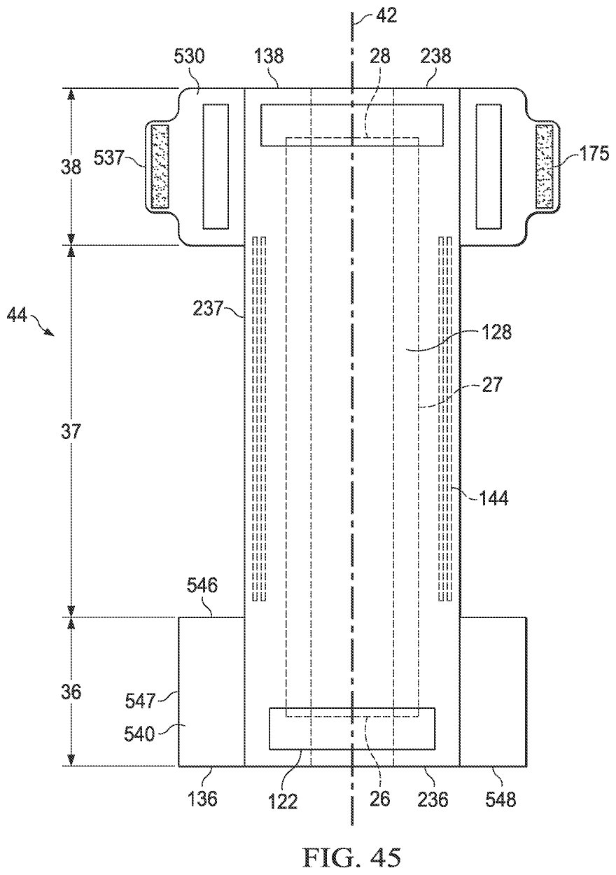

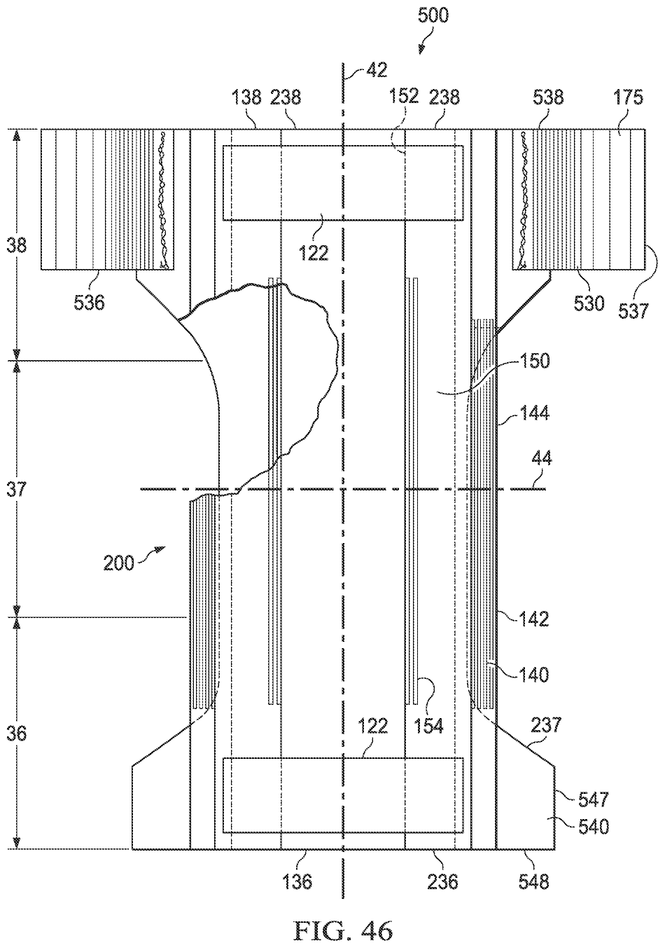

The taped diaper 500, open form article, as illustrated in FIGS. 38, 39, 44, 45, 46 and 47 may comprise elastomeric side flaps 530 in one or both of the front waist region 36 and back waist region 38. The elastomeric side flaps 530 may be formed (joined and/or positioned) in a particular place or position and may be unitary structurally with other elements of the article 100 or as a separate element joined to another element of the article 100. The elastomeric side flaps 530 provide an elastically extensible feature that provides a more comfortable and contouring fit by initially conformably fitting the article 100 to the wearer and sustaining this fit throughout the time of wear well past when the taped diaper 500 has been loaded with exudates since the elastomeric side flaps 530 allows the diaper to expand and contract to fit the wearer. Further, the elastomeric side flaps 530 develop and maintain wearing forces (tensions) and enhance the tensions developed and maintained by the fastening system 179, primary fasteners 175, to maintain the article 100 on the wearer and enhance the fit. The elastomeric side flaps 530 especially assist in maintaining the primary line of tension formed by the fastening system 179 allowing the diaper to conformably fit over the hips of the wearer where there is dynamic motion, and initially pretensioning the waist opening 190 and leg opening 192 since the diaperer typically stretches the elastomeric side flaps 530 when applying the taped diaper 500 on the wearer so that when the elastomeric side flaps 530 contract, tension is transmitted from the elastomeric side flaps 530 along the waist opening 190 and along at least a portion of the leg opening 192. The elastomeric side flaps 530 further provide more effective application of the diaper since even if the diaperer pulls one elastomeric side flap further than the other during application (asymmetrically), the diaper will “self-adjust” during wear. While the open form article of the present disclosure may have the elastomeric side flaps 530 disposed in the back waist region 38, alternatively, the taped diaper 500 may be provided with elastomeric side flaps 530 disposed in the front waist region 36 or in both the front waist region 36 and the back waist region 38. The open form article may also have elastomeric side flaps 530 disposed in a first waist region and non-elastomeric side flaps or non-elastomeric ears 540 disposed in a second waist region.

The elastomeric side flaps 530 may be constructed in a number of configurations. For example, the elastomeric side flaps may 530 comprise a separate elastically extensible material or laminate joined to the chassis 200 as illustrated in FIGS. 38, 39, 45 and 46 or may be a unitary stretch laminate formed by operatively associating an elastomeric; material with the center chassis 200 as illustrated in FIG. 47. Examples of diapers with elastomeric side flaps useful in the present disclosure are disclosed in U.S. Pat. No. 4,857,067, entitled “Disposable Diaper Having Shirred Ears” issued to Wood, et al. on Aug. 15, 1989; U.S. Pat. No. 4,381,781 issued to Sciaffara, et al. on May 3, 1983; U.S. Pat. No, 4,938,753 issued to Van Gompel, et al. on Jul. 3, 1990; and the hereinbefore referenced U.S. Pat. No. 5,151,092 issued to Buell, et al. on Sep. 29, 1992; each of which are incorporated herein by reference.

In one embodiment as shown in FIGS. 38 and 45 the taped diaper 500, open form, may comprise a first elastomeric side flap 530 disposed in a first waist region and a second elastomeric side flap 530 disposed in a first waist region. The taped diaper may also comprise an elastomeric waistband 122 disposed in one or both waist regions. The taped diaper may also comprise a substantially rectangular chassis 200.

In addition to a pair of laterally opposing elastomeric side flaps 530 in a first waist region the tape diaper 500 may comprise a pair of laterally opposing discrete ears 540 disposed in a second waist region, wherein one or both of the side flaps 530 and the ears 540 are non-rectangular as shown in FIG. 38. In an alternative embodiment, illustrated in FIG. 39 the tape diaper may comprise a pair of laterally opposing elastomeric side flaps 530 disposed in a first waist region and a pair of laterally opposing integral ears 540 formed in part by a portion of one or more of the backsheet 125, topsheet 124, outer leg cuff 140, or landing zone and being disposed in a second waist region.

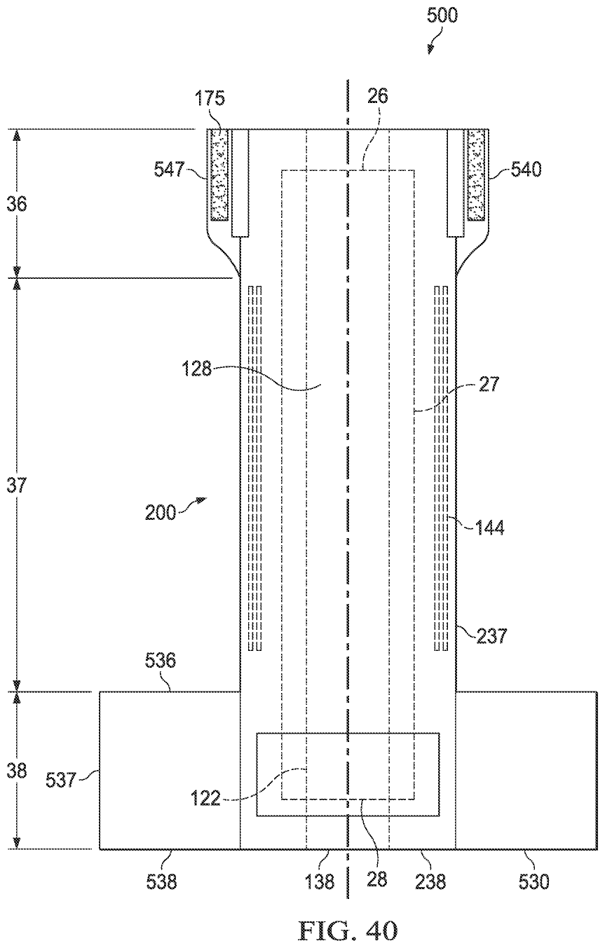

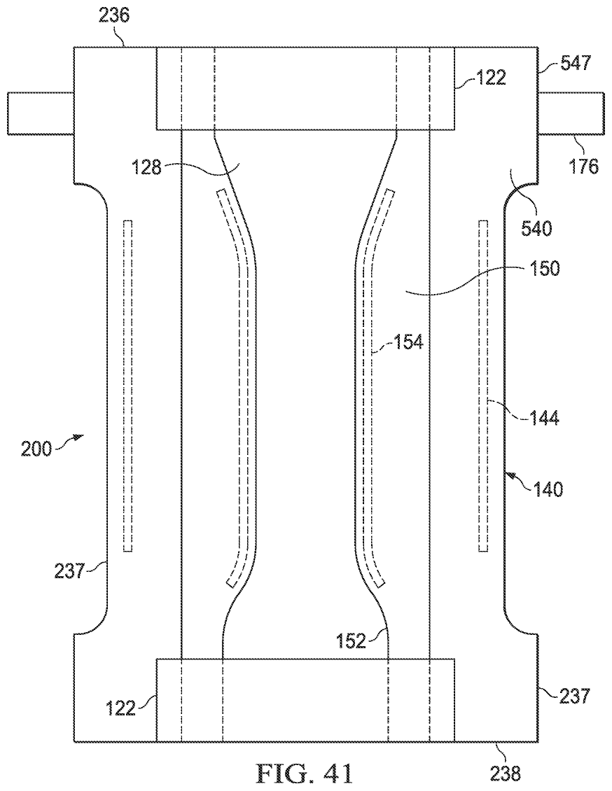

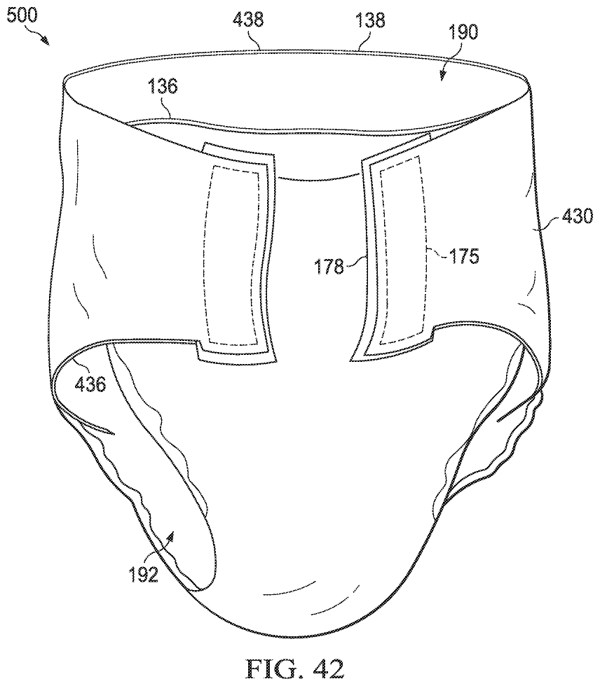

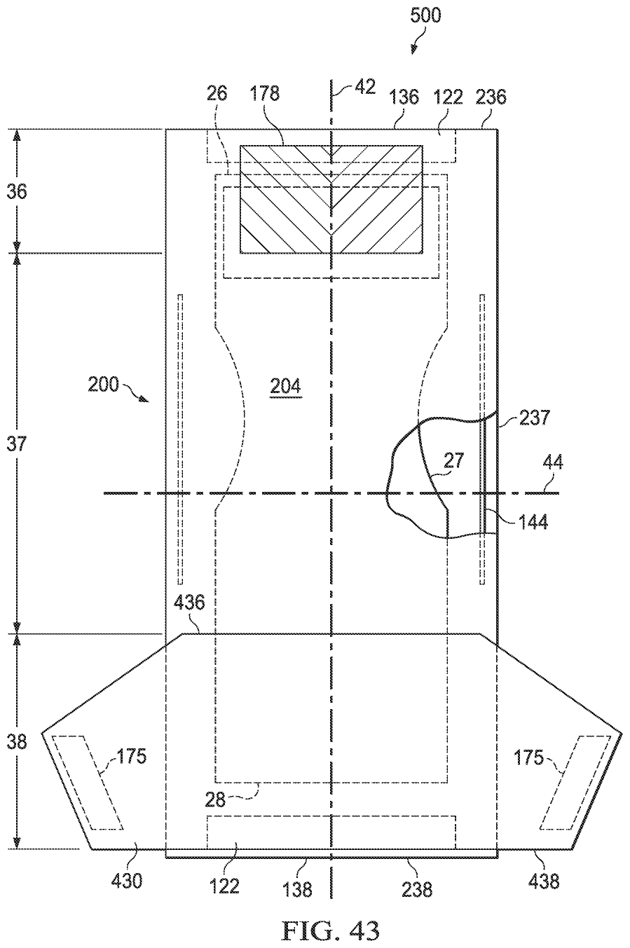

In an alternative embodiment the taped diaper 500 may comprise a first non-elastomeric ear 540 disposed in a first waist region and a second non-elastomeric ear 540 disposed in a first waist region and an elastomeric waistband 122 disposed in a first waist region as illustrated in FIGS. 40 and 41.

In certain embodiments, the elastomeric waistband 122 may have a lateral width that is >60% of a lateral width of the center chassis 200 and may comprise a pair of laterally opposing fasteners 175 disposed in the same waist region as the waistband 122. The taped diaper 500 may have a waistband 122 that may overlap portions of two or more of an absorbent core 128 and/or a topsheet 124 and/or the inner leg cuffs 150 as illustrated in FIG. 41.

In certain embodiments, the taped diaper 500 may comprise a pair of laterally opposing front ears 540 joined to the chassis 200 in the front waist region 36 and a pair of laterally opposing elastomeric side flaps 530 joined to the chassis 200 in the back waist region 38. The chassis 200 of the taped diaper 500 may be substantially rectangular as shown in FIGS. 38 and 45. Alternatively the chassis 200 may be non-rectangular, e.g. a portion of the laterally opposing side edges 237 or a portion of the longitudinally opposing end edges 136 and 138 of the chassis may be notched or curved either convexly or concavely as shown in FIG. 39 and FIG. 47.

The side panel pant 300, closed form article, may comprise elastomeric side panels 330 in one or both of the front waist region 36 and back waist region 38. The elastomeric side panels 330 may be formed (joined and/or positioned) in a particular place or position and may be unitary structurally with other elements of the article or as separate discrete elements joined to another element of the article. In certain embodiments having front and back elastomeric side panels 330, the front and back side panels 330 on one side of the article may be joined permanently or refastenably to each other and the front and back side panels 330 on the opposing side of the article may be joined permanently or refastenably to each other to create a waist opening 190 and a pair of leg openings 192. The elastomeric side panels 330 provide an elastically extensible feature that provides a more comfortable and contouring fit by initially conformably fitting the article 100 to the wearer and sustaining this fit throughout the time of wear well past when the pant has been loaded with exudates since the elastomeric side panels 330 allow the sides of the pant to expand and contract. Further, the elastomeric side panels 330 provide ease of application and develop and maintain wearing forces and tensions to maintain the article 100 on the wearer and enhance the fit. The elastomeric side panels 330 enable ease of application allowing the pant to be pulled conformably over the hips of the wearer and positioned at the waist where the side panels 330 conform to the body and provide tension sufficient to maintain the articles position on the wearer. The tension created by the side panels 330 is transmitted from the elastic side panels 330 along the waist opening 190 and along at least a portion of the leg opening 192. Typically the chassis 200 is disposed between the side panels 330 and extends to form a portion of the waist edge 136 and/or 138 of the side panel pant 300. In other words a portion of the waist edge 136 and/or 138 in one or both of the front waist region 36 and back waist region 38 may be formed in part by the side panels 330 and in part by the chassis 200. The side panels 330 can be integral with a portion of the chassis 200 or may discrete elements that overlap a portion of the chassis 200 and are joined thereto. The side panels 330 may be formed in part with an elastomeric film providing a film side panel having a first modulus or may be formed in part with elastomeric strands providing a stranded side panel having a second modulus wherein the modulus of the film side panel is greater than the modulus of the stranded side panel.

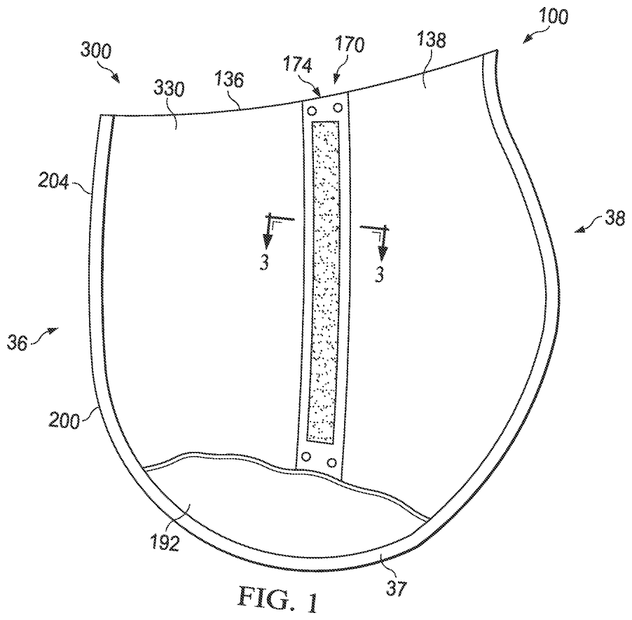

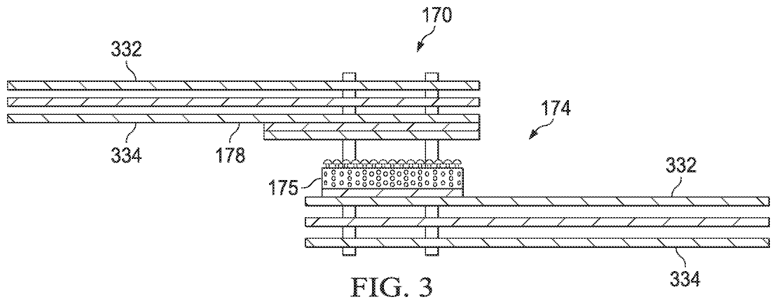

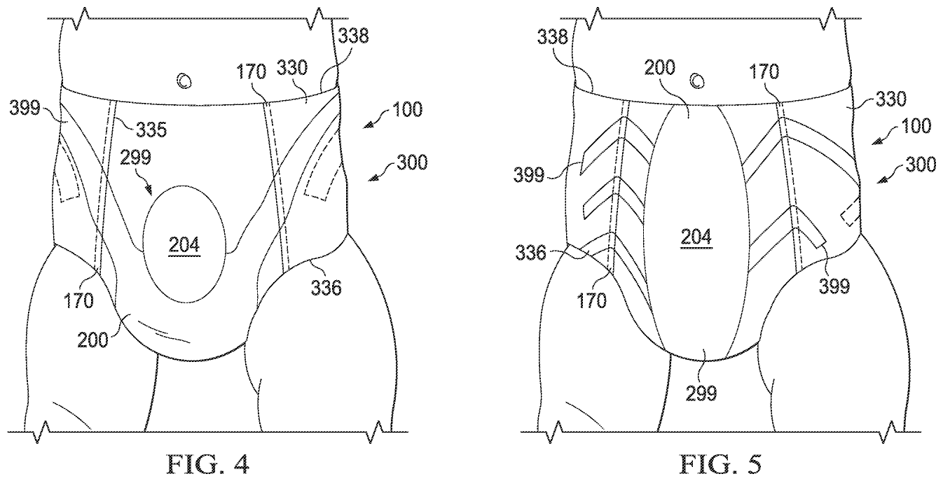

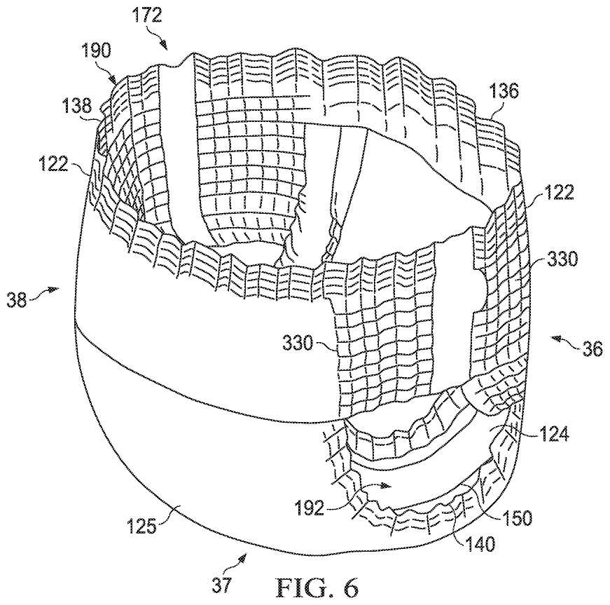

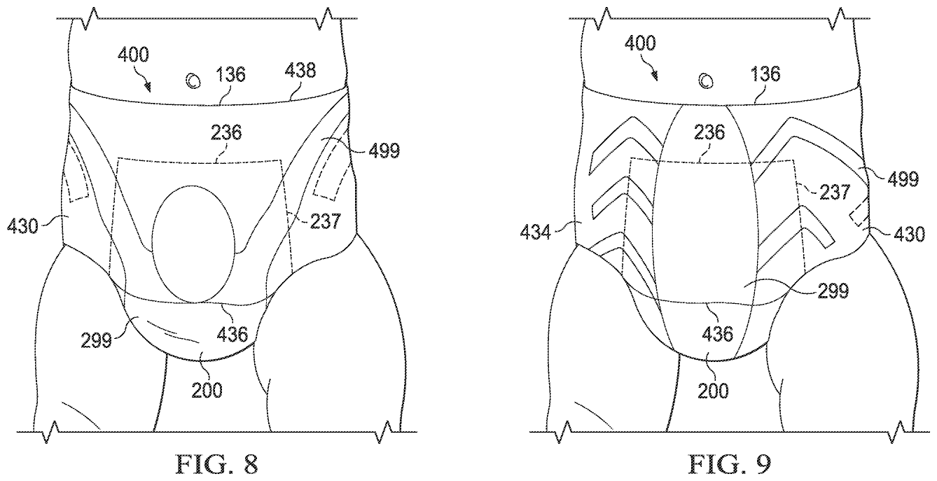

Disposable absorbent side panel pants 300 are one of the key forms of pants in the arrays of the present disclosure. The side panel pants 300 may be formed into a pant in a number of ways. Referring to FIGS. 4, 5, 8 and 9, the side panel pant 300 may comprise a first discrete elastomeric side panel 330 which may have a first side panel graphic 399 disposed thereon and a second laterally opposing discrete elastomeric side panel 330 which may have a second side panel graphic 399 disposed thereon. The graphics disposed on one or both of the elastomeric side panels 330 may be substantially aligned with a chassis graphic 299 disposed on the chassis. The discrete elastomeric side panels 330 may be disposed in one or both of the waist regions. The side panel pant 300 may also comprise a pair of laterally opposing refastenable seams 174 as illustrated in FIGS. 1-3. The refastenable side seam 174 may be formed by refastenably joining an interior surface of a portion of the article, e.g. a side panel 330, to an exterior surface of another portion of the article 100, e.g. a longitudinally opposing side panel 330 or the chassis 200 to form the refastenable side seam 174. In an alternative embodiment the side panel pant 300 may comprise a first seam 170 that is permanent and a fastener 175 disposed at or adjacent the permanent seam 172 such that when the permanent seam 172 is opened the fastener 175 may be exposed thereby enabling the fastener 175 to be attached to the article 100 to form a refastenable seam 174. Alternatively the side panel pant 300 may comprise a first seam that is permanent as described above and a perforation disposed on a side panel 330 and a fastener 175 disposed at or adjacent the perforation such that when the perforation is opened the fastener 175 is exposed thereby enabling the fastener 175 to be attached to the article 100 to form a refastenable seam 174. The refastenable seams 174 may comprise a fastening system 179 comprising a primary fastener 175, for example a mechanical fastener, velcro-like fasteners, hooks, etc., or mating fastener 178, for example a nonwoven or loop material intended to mate with a hook fastener, disposed on one or more of the discrete elastomeric side panels 330, for example a first portion of a fastening system 179 may be disposed on a first discrete elastomeric side panel 330 and a second portion of a fastening system 179 may be disposed on a second discrete elastomeric side panel 330. Alternatively, the side panel pant 300 may also comprise a first permanent side seam 172 and a laterally opposing second permanent side seam 172 as illustrated in FIGS. 6-9. The permanent side seam 172 may be formed by joining an interior surface of a portion of the article 100, e.g. a side panel 330, to an exterior surface of another portion of the article 100, e.g. a longitudinally opposing side panel 330 or the chassis 200 to form the permanent side seam 172. Alternatively, the permanent side seam 172 may be formed by joining an interior surface of a portion of the article 100, e.g. a side panel 330, to an interior surface of another portion of the article 100, e.g. a longitudinally opposing side panel 330 to form the permanent side seam 172. Any of the side panel pant 300 configurations described above may comprise a waistband 122 wherein at least a portion of the waistband 122 is disposed at or immediately adjacent the waist edge 136 and/or 138 and overlaps a portion of the center chassis 200. The waistband 122 may extend laterally to overlap portions of the inner leg cuffs 150 and/or portions of the elastomeric side panels 330. The waistband 122 may be disposed on the interior surface 202 of the chassis 200 or alternatively between the topsheet 124 and the backsheet 125.

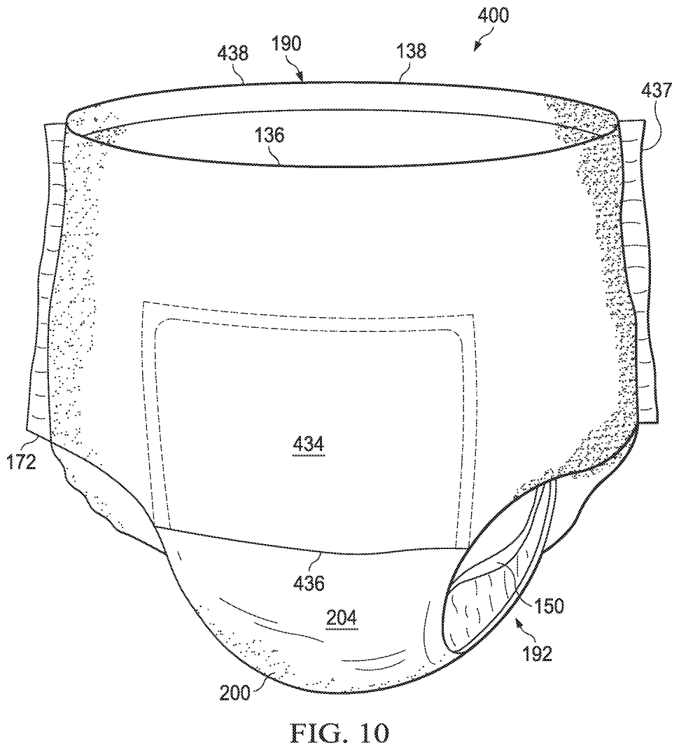

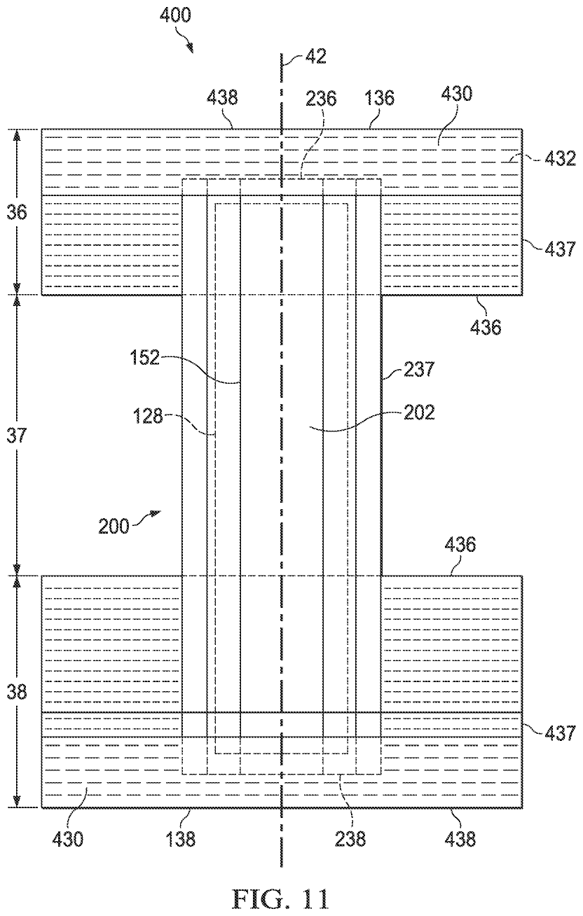

As illustrated in FIGS. 10 and 11, the belt flap pant 400, closed form article, may comprise elastomeric belt flaps 430 in one or both of the front waist region 36 and back waist region 38. The elastomeric belt flaps 430 may be formed (joined and/or positioned) in a particular place or position and may be unitary structurally with other elements of the article 100 or as separate discrete elements joined to another element of the article 100. In certain embodiments having front and back elastomeric belt flaps 430, the belt flap 430 in one waist region of the article may be joined permanently or refastenably to the belt flap 430 in the opposing waist region of the article to create a waist opening 190 and a pair of leg openings 192. The elastomeric belt flaps 430 provide an elastically extensible feature that provides a more comfortable and contouring fit by initially conformably fitting the article 100 to the wearer and sustaining this fit throughout the time of wear well past when the article has been loaded with exudates since the elastic belt flaps 430 allow the waist and sides of the diaper to expand and contract. Further, the elastic belt flaps 430 provide ease of application and develops and maintains wearing forces and tensions to maintain the article 100 on the wearer and enhance the fit. The elastomeric belt flaps 430 enable ease of application allowing the pant to be pulled conformably over the hips of the wearer and positioned at the waist where the belt flaps 430 conform to the body and provide tension sufficient to maintain the articles position on the wearer. The tension created by the belt flaps 430 is transmitted from the elastic belt flaps 430 along the waist opening 190 and along at least a portion of the leg opening 192. The elastomeric belt flaps 430 may be formed in part with elastic strands providing a stranded belt flap having a belt modulus wherein belt modulus of the stranded belt flap is less than the modulus of a film side panel 330 described herein. This difference in moduls enables extension of the elastomeric belt flap 430 at a lower force that that of an elastomeric film side panel 330 thereby making application of the belt flap pant 400 by a wearer easier than a side panel pant 300 enabling the wearer to develop skills valuable for their physiological and psychological development.

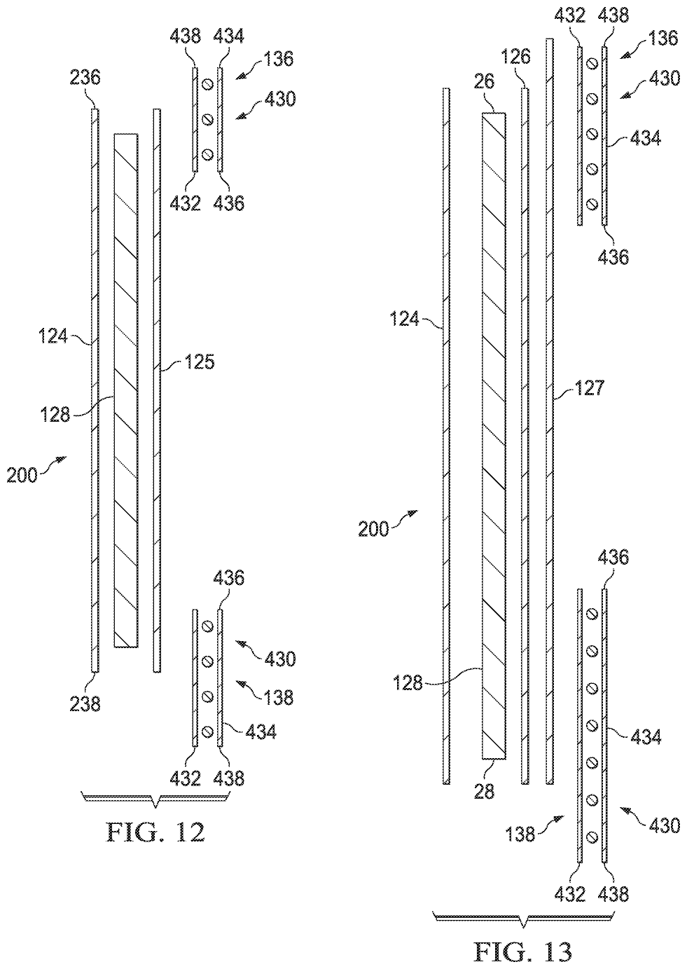

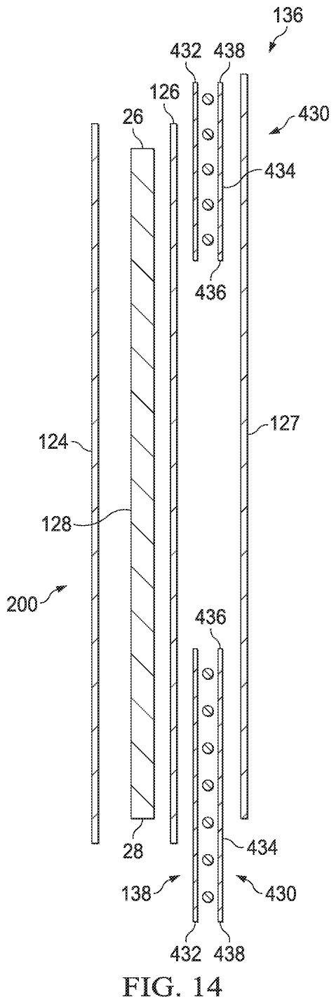

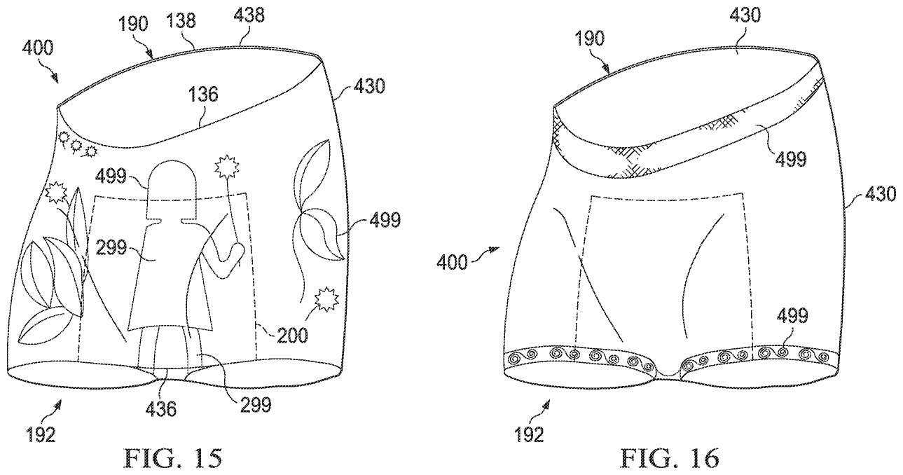

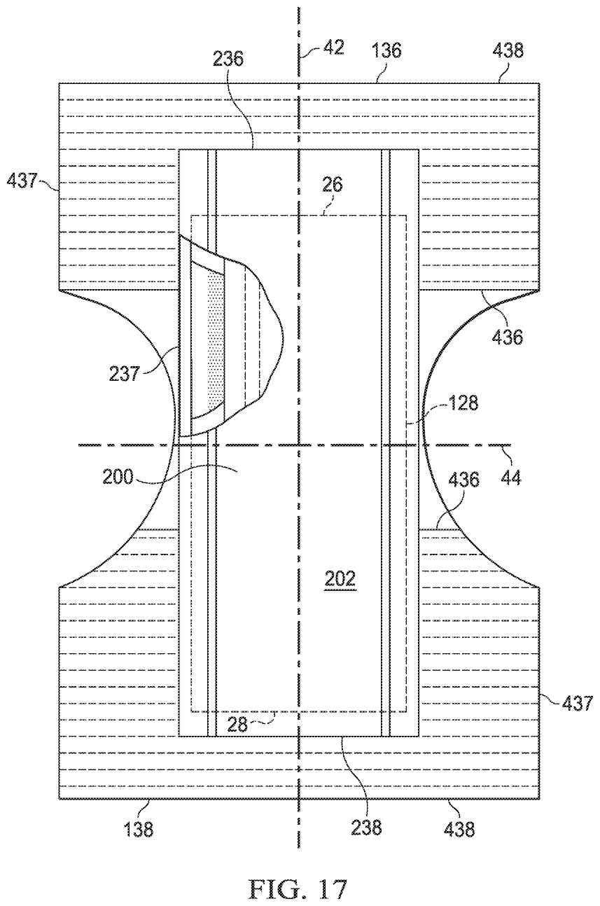

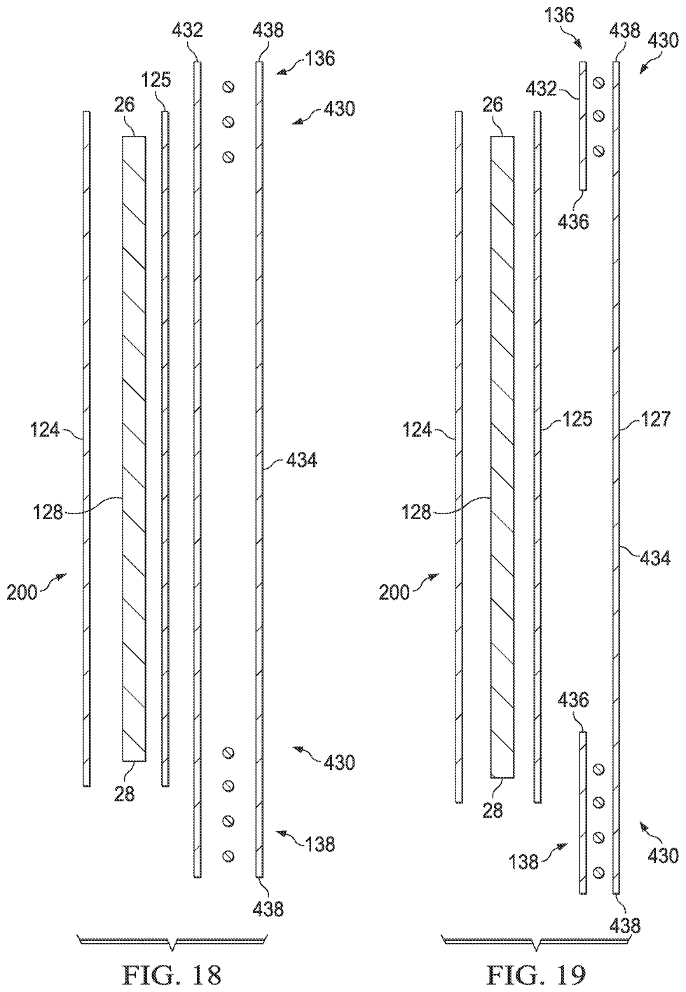

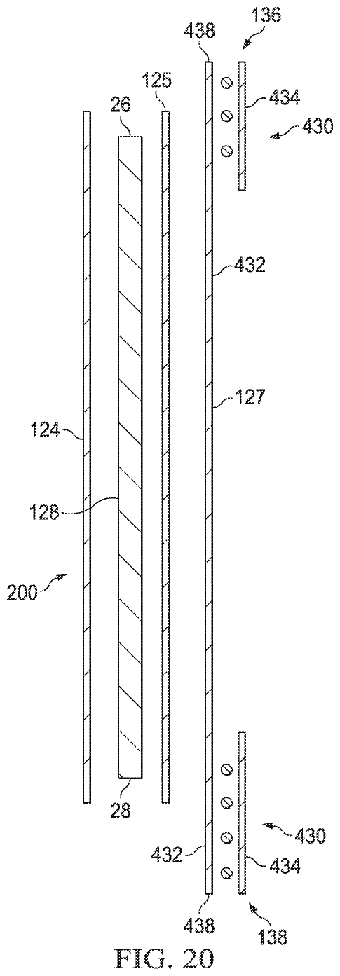

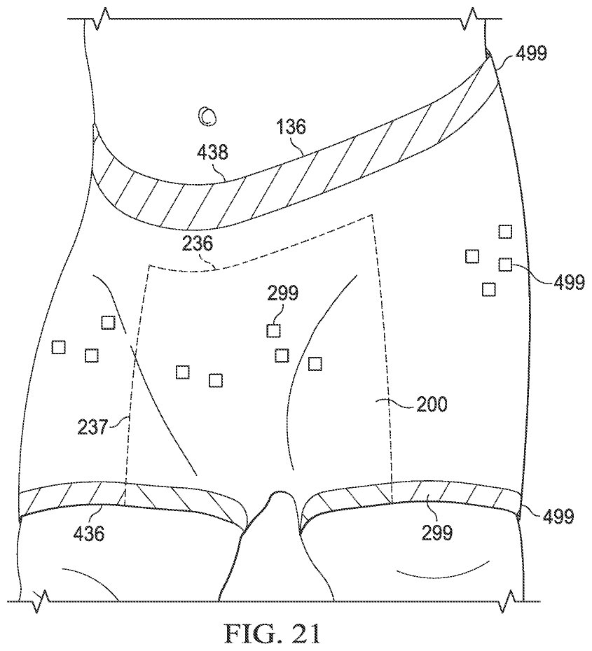

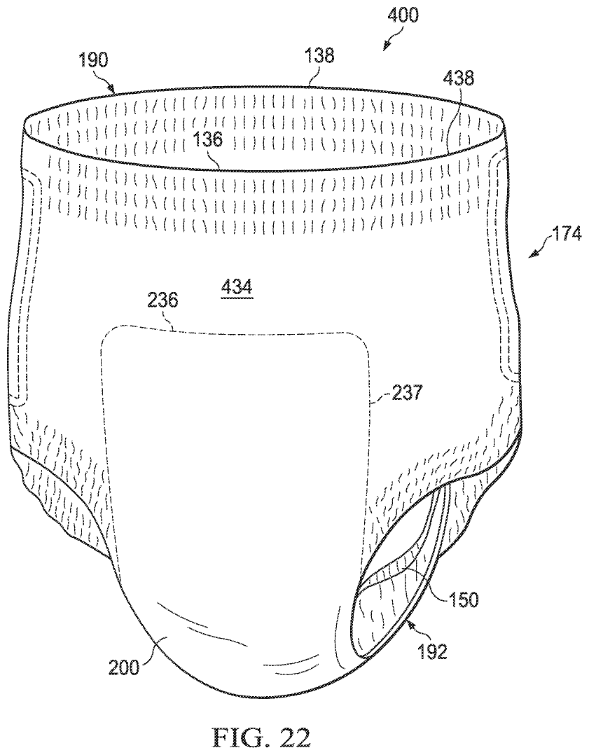

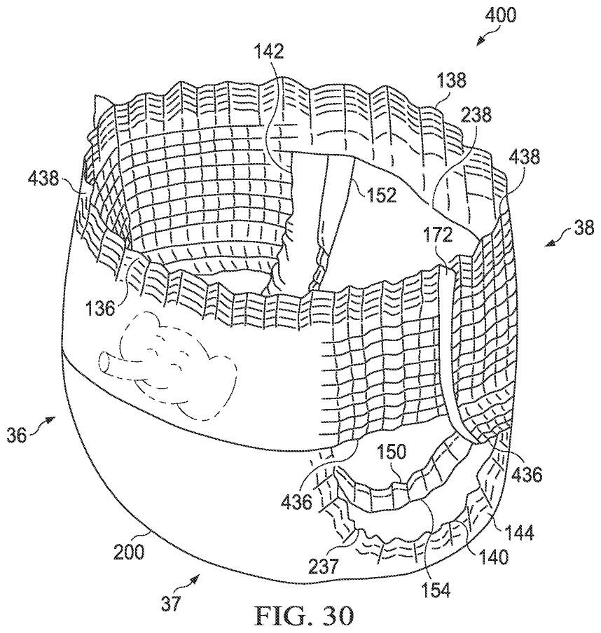

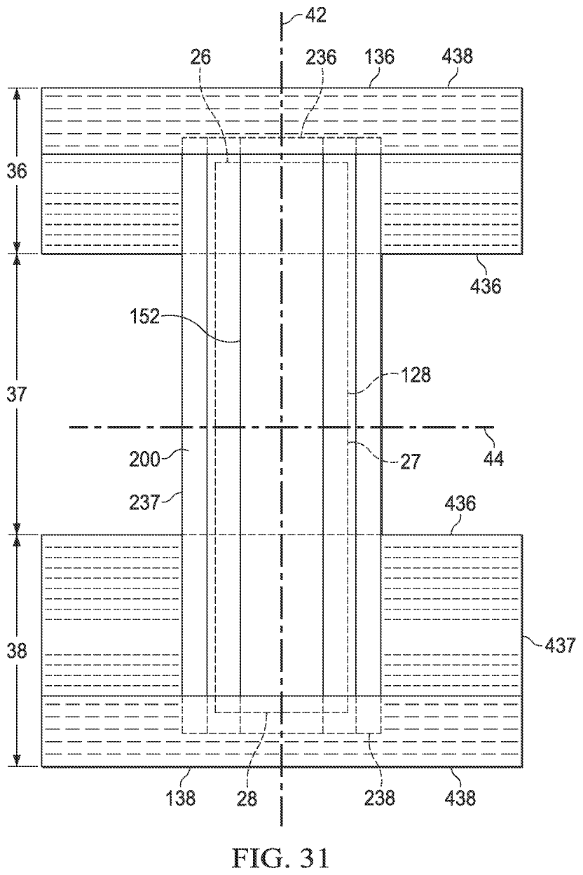

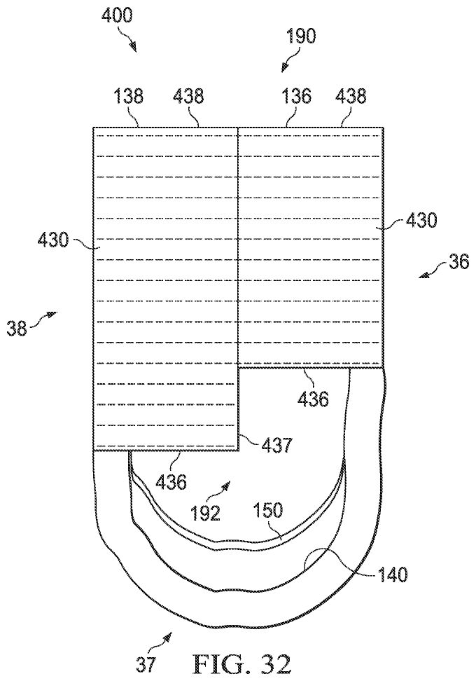

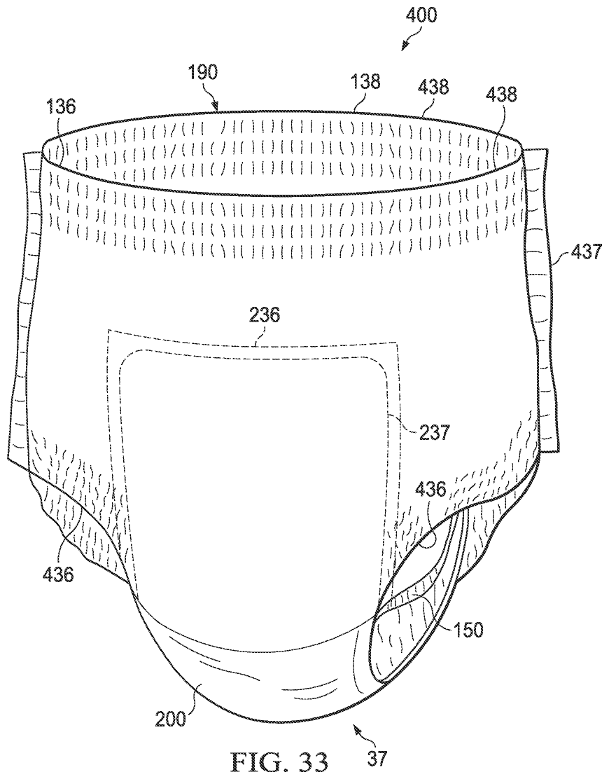

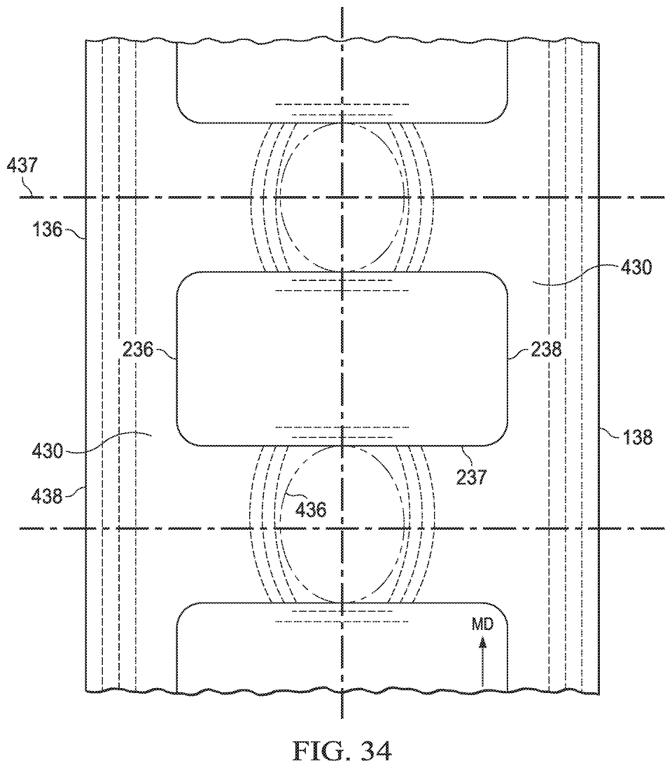

Disposable absorbent elastomeric belt flap pants 400 are one of the key forms of pants in the arrays of the present disclosure. As illustrated in FIGS. 15, 16 and 21, the belt flap pant 400 may comprise a first elastomeric belt flap 430 which may comprise a first belt flap graphic 499 and a second elastomeric belt flap 430 which may also comprise a second belt flap graphic 499. The first and second belt flap graphics 499 may be substantially aligned with each other along the side edges 437 of the respective belt flaps 430. In some embodiments the belt flap graphic 499 on one or both of the first elastomeric belt flaps 430 may be substantially aligned with a chassis graphic 299 disposed on a chassis 200. In certain embodiments, one or both of the elastomeric belt flaps 430 may comprise one or more of an arrangement of belt flap graphics covering greater than about 30% of the surface area of the belt flap 430 and/or an arrangement of belt flap graphics when viewed from the outside appear to comprise at least three colors and/or an arrangement of belt flap graphics being disposed within 30 mm of a waist edge 438 of the belt flap 430 and/or an arrangement of belt graphics being disposed within about 10 mm of a leg edge 436 of the belt flap 430 as illustrated in FIGS. 15 and 16. The belt flap graphics 499 play a pivotal role in the psychological development of the child as they provide the appearance of real underwear. In certain embodiments, one or both of the elastomeric belt flaps 430 may comprise one or more of a belt flap graphic 499 and/or a colored nonwoven and/or a tinted nonwoven. As shown in FIGS. 10-14, the first and second elastomeric belt flaps 430 may be discrete and longitudinally opposed, wherein the first elastomeric belt flap 430 is disposed in a first waist region and the second elastomeric belt flap 430 is disposed in a second waist region. The longitudinally opposed belt flaps 430 may be joined along the side edges 437 of the belt flaps 430 by permanent side seams 172. The permanent side seam 172 may be formed by joining an interior surface of a portion of the article 100, e.g. a belt flap 430, to an exterior surface of another portion of the article 100, e.g. a longitudinally opposing belt flap 430 or the chassis 200 to form the permanent side seam 172. Alternatively, the permanent side seam 172 may be formed by joining an interior surface of a portion of the article 100, e.g. a belt flap 430, to an interior surface of another portion of the article 100, e.g. a longitudinally opposing belt flap 430 to form the permanent side seam 172. Alternatively, as shown in FIGS. 22-29 the longitudinally opposed belt flaps 430 may be joined along the side edges 437 of the belt flaps 430 by refastenable side seams 174. The refastenable side seam 174 may be formed by refastenably joining an interior surface of a portion of the article 100, e.g. a belt flap 430, to an exterior surface of another portion of the article 100, e.g. a longitudinally opposing belt flap 430 or the chassis 200 to form the refastenable side seam 174. Alternatively the refastenable side seam 174 may comprise an intermediate member 171 which may be permanently or refastenably joined to one of the belt flaps 430 and the intermediate member 171 is refastenably joined to one of the interior surface or exterior surface of a portion of the article 100, e.g. the opposing belt flap 430 or the chassis 200 to form the refastenable side seam 174. In an alternative embodiment a belt flap pant 400 may comprise a first seam that is permanent as described above and a fastener 175 disposed at or adjacent the permanent seam 172 such that when the permanent seam 172 is opened the fastener 175 may be exposed thereby enabling the fastener 175 to be attached to the article 100 to form a refastenable seam 174. Alternatively the belt flap pant 400 may comprise a first seam that is permanent as described above and a perforation disposed on a belt flap 430 and a fastener 175 disposed at or adjacent the perforation such that when the perforation is opened the fastener 175 may be exposed thereby enabling the fastener 175 to be attached to the article 100 to form a refastenable seam 174. In an alternative embodiment one or both of the first and second elastomeric belt flaps 430 may comprise an inner belt flap layer 432 and an outer belt flap layer 434. In certain embodiments the inner belt flap layer 432 and/or the outer belt flap layer 434 of the first and second elastomeric belt flaps 430 may be formed by a common belt flap layer as shown in FIGS. 17-21. In embodiments where the first and second elastomeric belt flaps 430 have a common belt flap layer, the common belt flap layer may extend from a first waist edge in a first waist region to a longitudinally opposing second waist edge in a second waist region, i.e. front waist edge 136 to back waist edge 138. The belt flap pant 400 may have a first elastomeric belt flap 430 disposed in a first waist region having a first longitudinal length and a second elastomeric belt flap 430 disposed in a second waist region having a second longitudinal length wherein the longitudinal length of the first belt flap is greater than the longitudinal length of the second belt flap along the side edge of the belt flap at or adjacent the side seam as illustrated in FIGS. 11, 13, 30, 31 and 32. This length difference helps provide buttock coverage in the back of the pant providing a more underwear-like appearance. The belt flap pant 400 may also comprise a first refastenable seam 174 and a second refastenable seam 174 joining the first elastomeric belt flap 430 and the second elastomeric belt flap 430 to form a waist opening 190 and a pair of leg openings 192. The belt flap pant 400 may also comprise a temperature change agent to provide a temperature change sensation for the wearer whenever the pant is exposed to exudates such as urine. The belt flap pant 400 may comprise belt flaps 430 having apertured nonwovens forming one or both of the inner belt layer 432 or outer belt layer 434. The apertured nonwoven belt flap layers provide increased breathability as well as increased softness and a more garment-like feel.

An absorbent article 100 of the present array comprising a reusable, durable or semi-durable, chassis 600 and a removable and replaceable disposable absorbent insert 628 forming a two piece reusable chassis 600 and insert 628 system. The chassis 600 may be in the form of a pant having a pre-defined waist opening 190 and a pair of pre-defined leg openings 192 and a means for receiving a disposable absorbent insert 628. This form of product can transform durable underwear into an absorbent article 100 to address nocturnal enuresis giving the wearer the option of wearing the article with or without an absorbent insert. This form may also come in a taped, open form, diaper 500 for used on individuals who are physically and/or psychologically challenged and need substantial assistance changing their absorbent articles.

One or more of the absorbent articles described above may comprise a chassis 200 comprising one or more of a chassis graphic 299 disposed on the chassis 200 and/or a colored nonwoven and/or a tinted nonwoven.

### Arrays

The arrays of the present disclosure may comprise a plurality of different absorbent articles each being sized appropriately for wearers weighing less than 75 pounds. The arrays of the present disclosure comprise at least one belt flap pant 400 and two or more, i.e., three, four or five, packages each package comprising a different article 100 from the absorbent articles 100 described above. The array may also comprise at least one belt flap pant 400 and two or more, i.e., three, four or five packages having substantially overlapping weight ranges. A desirable array of the present disclosure may comprise a plurality of absorbent articles 100 comprising at least one belt flap pant 400 and one or more side panel pants 300 and one or more taped diapers 500 wherein the absorbent articles 100 in the array have one or more of the same brand name and/or the same sub-brand name and/or are made by or for the same manufacturer and/or have commonly assigned trademarks wherein the array is available at a common point of sale, e.g. online website, retail establishment and/or located in proximity to each other within a retail establishment and/or in proximity to each other on a common shelf display.

In some embodiments, arrays of the present disclosure may comprise one or more articles 100 comprising a wetness indicator. The wetness indicator being designed to change color and/or appear and/or disappear when exposed to bodily exudates like urine and/or BM. The wetness indicator may be located on the interior surface of the backsheet 125 or alternatively on one or more layers disposed interiorly of the backsheet 125. The wetness indicator may be activated by the pH of the exudates or by a pH change resulting from mixing of the exudates and a component in the diaper or alternatively may be water soluble when wetted with liquid exudates. The wetness indicator may form a portion of, may be substantially aligned with or may be coordinated with a graphic element on one or more of the chassis 200, side flap 530, side panel 330, or belt flap 430.

The array may also include one or more articles 100 having side panels 330, side flaps 530, belt flaps 430, chassis 200 or fasteners 175 that are shaped, i.e. non-rectangular, each being separately formed from a continuous web or laminate by cutting or severing the web along a nested pattern such that the shape of the side panel 330, side flap 530, belt flap 430, chassis 200 or fastener 175 disposed on one side of the cut line is substantially the same as the side panel 330, side flap 530, belt flap 430, chassis 200 or fastener 175 disposed on opposing side of the cut line such that when placed together they fit substantially into each other.

The array of the present disclosure wherein one or more of the articles 100 may comprise an extensible, desirably elastomerically extensible, nonwoven disposed on one or more of the chassis 200, side panel 330, side flap 530 or belt flap 430.

The array of the present disclosure wherein one or more of the pant articles may comprise a graphic indicating the front and/or back of the pant article.

### Topsheets

The absorbent articles 100 of the present disclosure may comprise a topsheet 124. The topsheet 124 may be compliant, soft feeling, and non-irritating to the wearer's skin and may be elastically stretchable in one or more directions. Further, the topsheet 124 may be liquid pervious, permitting liquids (e.g., menses, urine, and/or runny feces) to penetrate through its thickness. Various topsheets may also comprise a hydrophilic material, for example, which is configured to draw bodily fluids into an absorbent core 128 of the chassis 200 when these fluids are expelled from the body. A suitable topsheet 124 may be manufactured from a wide range of materials, such as porous foams, reticulated foams, apertured plastic films, or woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. If the topsheet 124 includes fibers, the fibers may be spunbond, carded, wet-laid, meltblown, hydroentangled, or otherwise processed as is known in the art, in particular spunbond PP nonwoven, for example. Suitable apertured films may comprise those described in U.S. Pat. Nos. 3,929,135, 4,324,246, 4,342,314, 4,463,045, 5,006,394, 5,628,097, 5,916,661, 6,545,197, and 6,107,539.

The topsheet 124 may be joined to the backsheet 125, the core 128 and/or any other layers as is known to those of skill in the art. Usually, the topsheet 124 and the backsheet 125 are joined directly to each other in some locations (e.g., on or close to the periphery of the article 100) and are indirectly joined together in other locations by directly joining them to one or more other elements of the absorbent article 100, e.g. absorbent core 128.

Apertured film or nonwoven topsheets are typically pervious to bodily exudates, yet non-absorbent, and have a reduced tendency to allow fluids to pass back through and rewet the wearer's skin. Suitable woven and nonwoven materials may comprise natural fibers, such as, for example, wood or cotton fibers, synthetic fibers, such as, for example, polyester, polypropylene, or polyethylene fibers, or combinations thereof. If the topsheet 124 comprises fibers, the fibers may be spunbond, carded, wet-laid, meltblown, hydroentangled, or otherwise processed, for example, as is generally known in the art.

The topsheet 124 may comprise a texture formed by geometrical treatments, morphilogical treatments, chemical treatments describe hereinafter. The topsheet 124 may also form a portion of a three dimensional substrate as described hereinafter.

The topsheet 124 may extend to the waist edge of the article 100 especially in those articles wherein the chassis 200 extends to the waist edge. Alternatively for products wherein the length of the chassis 200 is less than the length of the article 100 the topsheet 124 may extend only to the end of the chassis 200 and therefore not the waist edge of the article 100.

The topsheet 124 may comprise a skin care lotion. Examples of suitable lotions include, but are not limited to, those described in U.S. Pat. Nos. 5,607,760; 5,609,587; 5,635,191; 5,643,588; and 5,968,025, and as described in U.S. Application No. 61/391,353.

In one embodiment, the topsheet 124 may comprise graphics such that depth perception is created as described in U.S. Pat. No. 7,163,528.

Typical absorbent article topsheets have a basis weight of from about 5 gsm to about 50 gsm, from about 10 to about 35 gsm or from about 12 to about 30 gsm, but other basis weights are within the scope of the present disclosure.

Absorbent articles of the present disclosure may comprise three-dimensional, liquid permeable substrates forming a portion of or all of the topsheet 124 as described in U.S. Ser. Nos. 14/656,820; 14/680,394; and 14/680,426.

The topsheet 124 may also comprise topsheet graphics disposed on one or both surfaces of the topsheet 124. The topsheet graphics may be coordinated with graphics on other components of the article 100, e.g. chassis 200, side panel 330, side flap 530, belt flap 430, and backsheet 125. In addition, graphics may be disposed on the exterior of the article 100 that coordinate with the topsheet graphics to make the absorbent article 100 have the appearance of underwear wherein the inside has a similar pattern, color, etc. to the exterior of the article 100. The graphics may also highlight various structural elements within the article.

### Backsheets

The absorbent article 100 of the present disclosure may comprise a backsheet 125. The backsheet 125 may be impervious, or at least partially impervious, to fluids or body exudates (e.g., menses, urine, and/or runny feces) and may be manufactured from a thin plastic film, although other flexible liquid impervious materials may also be used. The backsheet 125 may prevent the body exudates or fluids absorbed and contained in an absorbent core 128 of the absorbent article 100 from wetting articles which contact the absorbent article 100, such as bedsheets, pajamas, clothes, and/or undergarments. The backsheet 125 may comprise a woven or nonwoven material, polymeric films such as thermoplastic films of polyethylene or polypropylene, and/or a multi-layer or composite materials comprising a backsheet film 126 and a backsheet nonwoven 127 (e.g., having an inner film layer and an outer nonwoven layer). A suitable backsheet 125 may comprise a polyethylene film having a thickness of from about 0.012 mm (0.5 mils) to about 0.051 mm (2.0 mils). Examples of polyethylene films are manufactured by Clopay Corporation of Cincinnati, Ohio, under the designation BR-120 and BR-121, and by Tredegar Film Products of Terre Haute, Ind., under the designation XP-39385.

One suitable material for the backsheet 125 can be a liquid impervious thermoplastic film having a thickness of from about 0.012 mm (0.50 mil) to about 0.051 mm (2.0 mils), for example including polyethylene or polypropylene. Typically, the backsheet 125 can have a basis weight of from about 5 g/m² to about 35 g/m². The backsheet 125 can be typically positioned adjacent the outer-facing surface of the absorbent core 128 and can be joined thereto. For example, the backsheet 125 may be secured to the absorbent core 128 by a uniform continuous layer of adhesive, a patterned layer of adhesive, or an array of separate lines, spirals, or spots of adhesive. Illustrative, but non-limiting adhesives, include adhesives manufactured by H. B. Fuller Company of St. Paul, Minn., U.S.A., and marketed as HL-1358J. An example of a suitable attachment device including an open pattern network of filaments of adhesive is disclosed in U.S. Pat. No. 4,573,986. Another suitable attachment device including several lines of adhesive filaments swirled into a spiral pattern is illustrated by the apparatus and methods shown in U.S. Pat. Nos. 3,911,173; 4,785,996; and 4,842,666. Alternatively, the attachment device may include heat bonds, pressure bonds, ultrasonic bonds, dynamic mechanical bonds, or any other suitable attachment device or combinations of these attachment devices.

In one embodiment, the backsheet 125 may be embossed and/or matte-finished to provide a more cloth-like appearance. Further, the backsheet 125 may permit vapors to escape from the absorbent core of the absorbent article 100 (i.e., the backsheet 125 is breathable) while still preventing, or at least inhibiting, fluids or body exudates from passing through the backsheet 125. Example breathable materials may include materials such as woven webs, nonwoven webs, composite materials such as film-coated nonwoven webs, microporous films, and monolithic films. In one embodiment, the size of the backsheet 125 may be dictated by the size of the absorbent article 100 and the design or configuration of the absorbent article 100 to be formed, for example.

The backsheet 125 may be joined to the topsheet 124, the absorbent core 128, and/or any other element of the absorbent article 100 by any attachment methods known to those of skill in the art.

A portion or the whole of the chassis 200 may be made extensible to a degree greater than the inherent extensibility of the material or materials from which the chassis 200 is made, e.g., the backsheet 125, the topsheet 124, or both. The additional extensibility may be desirable in order to allow the chassis 200 to conform to the body of a wearer during movement by the wearer. The additional extensibility may also be desirable, for example, in order to allow the user of an article 100 including a chassis 200 having a particular size before extension to extend the front waist region 36, the back waist region 38, or both waist regions of the chassis 200 to encircle the waist of an individual wearer whose waist circumference falls within a predefined range, i.e., to tailor the fit to the individual wearer. Such extension of the waist region or regions may give the article 100 a generally hourglass shape, so long as the crotch region 37 is extended to a relatively lesser degree than the waist region or regions, and may impart a tailored appearance to the article 100 when it is worn.

Additional extensibility in the chassis 200 in the lateral direction is relatively more useful than additional extensibility in the longitudinal direction. The abdomen of the wearer is likely to expand when the wearer changes posture from standing to sitting and the corresponding abdominal expansion increases the circumference that is encircled by the waist edges of the chassis 200, necessitating the lateral extension of the waist region or regions.

Additional lateral extensibility in the chassis 200 may be provided in a variety of ways. For example, all or a portion of the chassis 200 may be made of a formed web material or a formed laminate of web materials like those described in U.S. Pat. No. 5,518,801 issued on 21 May 1996 in the name of Chappell et al.

Such a formed web material can be laterally extended beyond its original dimension with the application of a force. In particular, the effects of an application of opposing divergent forces will result in an extension of such a formed web material along an axis between the opposing forces and result in the generation of a resistive contractive force. Thus, such formed web materials exhibit an extensible behavior resembling that of traditional elastic materials in the range of extensibility that is useful for the type of lateral extension desired for use in absorbent articles.

The range of extensibility of the materials used in the chassis 200 of the absorbent article 100 may typically be formed to provide any range of extensibility from near zero to a maximum of more than 100 percent of the original dimension. In some embodiments of the present disclosure, a portion of the chassis 200 may have a maximum extensibility of approximately 20 percent of its original dimension. However, any particular value for the maximum extensibility in the range from approximately one percent to approximately 100 percent may be selected to suit a particular choice of the original size of the article 100 and the range of sizes of the intended wearers. In particular, a diaper having a specific unextended waist opening 190 circumference may be suitable for use on wearers having waist circumferences ranging from equal to this unextended waist opening 190 circumference up to the maximum extensibility.

Any of a variety of materials may be formed as described in the Chappell et al. '801 patent. For example, a film, a nonwoven, or a laminate of either or both of these materials may be formed to provide the desired extensibility. It is also possible to modify such a material in more than one way while forming it to provide extensibility. For example, a film that is originally formed to resist the permeation of vapor through its thickness and to contain fine particles of a granular filler material such as calcium carbonate may be treated as described in the Chappell et al. '801 patent to simultaneously provide extensibility and create small holes that allow water vapor to pass through its thickness. Thus, the film can simultaneously be rendered extensible and breathable.

### Leg Cuffs

The chassis 200 of the absorbent article 100 may comprise longitudinally extending and laterally opposing inner leg cuffs 150 and longitudinally extending and laterally opposing outer leg cuffs 140 that are disposed on the interior surface of the chassis 200 that faces inwardly toward the wearer and contacts the wearer. The inner leg cuffs 150 may comprise one or more elastic gathering members 154 disposed at or adjacent the proximal edge 152 of one or both of the inner leg cuffs 150. The outer leg cuffs 140 may comprise one or more elastic gathering members 144 disposed at or adjacent the distal edge 142 of one or both of the outer leg cuffs 140. The elasticized leg cuffs 140 and 150 may comprise several embodiments for reducing the leakage of body exudates or fluids in the leg regions. The elasticized leg cuffs are sometimes referred to as leg bands, barrier cuffs, elastic cuffs, or gasketing cuffs. Suitable elasticized leg cuffs may comprise those described in U.S. Pat. Nos. 3,860,003, 4,909,803, 4,695,278, 4,795,454, 4,704,115, and 4,909,803, and U.S. Pat. Publ. No. 2009/0312730. The inner and/or outer leg cuffs may be formed by folding portions of the chassis 200 laterally inward, i.e., toward the longitudinal axis 42, to form both the leg cuffs and the side edges 237 of the chassis 200. In other embodiments, the leg cuffs may be formed by attaching an additional layer or layers to the chassis 200 at or adjacent to each of the respective side edges 237 of the chassis 200. In one embodiment, the chassis 200 may also comprise other elastics disposed adjacent the side edges 237 which may cause the article 100 to form into a “U” shape when allowed to relax thereby pulling the chassis interior surface 202 of the front waist region 36 toward the chassis interior surface 202 of the back waist region 38.

In one embodiment, the inner leg cuffs 150 may comprise a proximal edge 152. The proximal edge 152 is positioned proximate to the longitudinal axis 42 compared to the side edges 237 of the chassis 200. The inner leg cuffs 150 may overlap the absorbent core 128, i.e., the proximal edges 152 lie laterally inward of the respective side edges 27 of the absorbent core 128. Such an overlapped configuration may be desirable in order to impart a more finished appearance to the absorbent article 100 than that imparted by a non-overlapped configuration. In other embodiments, the inner leg cuffs 150 may not overlap the absorbent core 128.

In one embodiment, the inner leg cuffs 150 may be attached to the interior surface 202 of the chassis 200 in a leg cuff attachment zone (not shown) adjacent to the front waist end edge 136 and in a longitudinally opposing leg cuff attachment zone (not shown) adjacent to the back waist end edge 138. In one embodiment, between the leg cuff attachment zones, the proximal edge 152 of the inner leg cuff 150 remains free, i.e., not attached to the chassis interior surface 202 of the chassis 200 or to the absorbent core 128. Also, between the longitudinally opposing inner leg cuff attachment zones, each leg cuff 150 may comprise one or more (specifically including one, two, three, or four elastic gathering members 154 per leg cuff 150) longitudinally extensible cuff elastic gathering members 154 that may be disposed at or adjacent to the proximal edge 152 of the inner leg cuff 150 by any suitable methods. Each of such inner cuff elastic gathering members 154 may be attached over the leg cuff's entire length or over only a portion of the leg cuff's length. For example, such inner cuff elastic gathering members 154 may be attached only at or near the leg cuff's longitudinally opposing ends and may be unattached at the middle of the leg cuff's length. Such inner cuff elastic gathering members 154 may be disposed in the crotch region 37 and may extend into one or both of the front waist region 36 and the back waist region 38. For example, an elastic gathering member 154 may be attached at or adjacent to the proximal edge 152 of each of the leg cuffs 150 and extend into both the front waist region 36 and the back waist region 38.

In various embodiments, each inner cuff elastic gathering member 154 may be enclosed inside a folded hem for example. In various embodiments, the inner cuff elastic gathering members 154 may be sandwiched between two layers forming the leg cuff 150, by two layers of the chassis 200, or may be sandwiched between a layer of the chassis 200 and a layer of one or both of the inner leg cuffs 150, and the topsheet 124.

In one embodiment, when stretched, the inner cuff elastic gathering member 154 disposed adjacent to each of the inner leg cuff's proximal edge 152 allows the leg cuff proximal edge 152 to extend to the flat uncontracted length of the chassis 200, e.g., the length of the chassis 200. When allowed to relax, the cuff elastic gathering member 154 contracts to pull the front waist region 36 and the back waist region 38 toward each other and, thereby, bend the article 100 into a “U” shape in which the interior of the “U” shape may be formed by the portions of the article 100 that are intended to be placed toward the body of the wearer (i.e., chassis interior surface 202). Because each of the proximal edges 152 remains free between the longitudinally oriented leg cuff attachment zones, the contractive force of the elastic gathering member 154 may lift the proximal edge 152 of the inner leg cuff 150 away from the interior surface 202 of the chassis 200. This lifting of the proximal edges 152 when the article 100 is in the relaxed condition lifts the leg cuffs 150 into a position to serve as side barriers to prevent, or at least inhibit, leakage of bodily exudates.

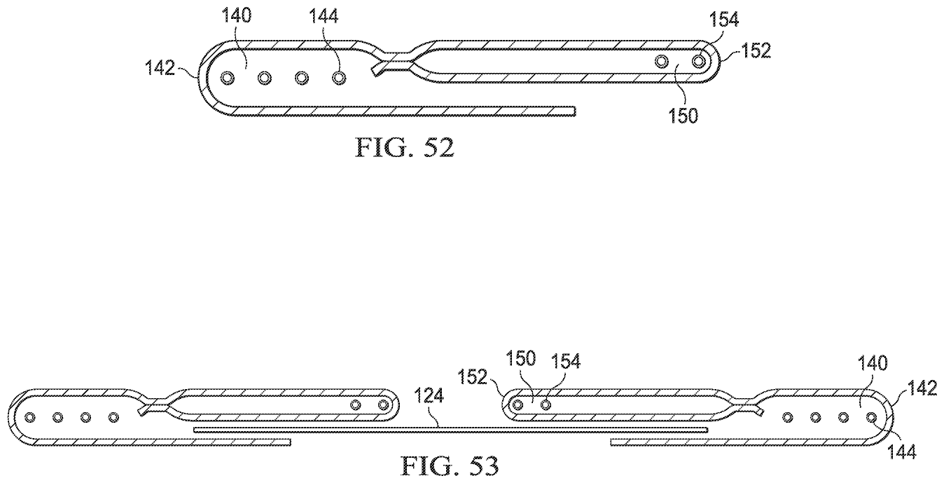

In certain embodiments, the absorbent article 100 may comprise an outer leg cuff 140 formed by a film, e.g. the backsheet film 126, a nonwoven, and an elastic material disposed between the backsheet film 126 and the nonwoven at or adjacent the side edge 237 of the chassis 200. In an alternative embodiment as illustrated in FIGS. 52-54, the absorbent article 100 may comprise an inner leg cuff 150 and/or an outer leg cuff 140 wherein one or both of the inner leg cuff 150 the outer leg cuff 140 may be formed from a folded nonwoven web and comprise inner cuff elastics 154 and outer cuff elastics 144 disposed between layers of the folded nonwoven web. Wherein a first fold in the folded nonwoven web forms the proximal edge 152 of the inner leg cuff 150 and a second fold in the folded nonwoven web forms the distal edge 142 of the outer leg cuff 140. In certain embodiments, the distal edge 142 of the outer leg cuff 140 may extend beyond the side edges of the backsheet and form a portion of the side edge 237 of the chassis 200 as illustrated in FIGS. 39 and 46. In such embodiments having a portion outer leg cuff 140 formed by a folded nonwoven web with an elastic material 144 disposed between layers of the folded nonwoven and extending outwardly from the side edges of the backsheet 125 forms a portion of the side edge 237 of the chassis 200 providing a finished more cloth-like outer cuff thereby providing a more underwear-like appearance.

In certain embodiments the outer leg cuff may be formed in part by a portion of the backsheet 125 e.g. the backsheet film 126 and/or backsheet nonwoven 127 wherein the elastic material 144 of the outer cuff is disposed between two layers formed by one or more of the backsheet film 126 and the backsheet nonwoven 127. In alternative embodiments the outer leg cuff may be formed from the backsheet nonwoven 127 and elastic material 144 wherein the elastic material 144 is disposed between two layers of the backsheet nonwoven 127. In certain executions the backsheet nonwoven 127 may be folded to encase the elastic material 144.

One or more of the inner leg cuffs 150 and outer leg cuffs 140 may comprise a multi-layer layer nonwoven material extending over the lateral extent of one or both of the inner leg cuff 150 and outer leg cuff 140.

### Waistbands

One or more of the absorbent article forms of the array may also comprise an elastomeric waistband 122 in addition to flaps/panels (side flaps, side panels) one or both of the front waist region 36 and back waist region 38. The elastomeric waistband 122 may be formed (joined and/or positioned) in a particular place or position and may be unitary structurally with other elements of the article 100 or as a separate element joined to another element of the article 100. The elastomeric waistband 122 provides an elastically extensible feature that provides a more comfortable and contouring fit by initially conformably fitting the article 100 to the wearer and sustaining this fit throughout the time of wear well past when the article 100 has been loaded with exudates since the waistband 122 allows the waist of the article 100 to expand and contract. Further, the waistband 122 develops and maintains wearing forces (tensions) and enhances the tensions developed and maintained by the closure system and/or the flaps/panels to maintain the article on the wearer and enhance the fit. The elastomeric waistband 122 especially assists in maintaining the primary line of tension formed by the primary fastening system 179 and/or flaps/panels allowing the article to conformably fit around the waist of the wearer where there is dynamic motion, and initially pretensioning the waist since the article typically is stretched at the waist when applying the article 100 on the wearer so that when the waistband 122 contracts and tension is transmitted from the waistband 122 along the waist opening 190. While some of the articles of the present disclosure may have the elastomeric waistband 122 disposed in the back waist region 38, alternatively, the articles 100 may be provided with elastomeric waistbands 122 disposed in the front waist region 36 or in both the front waist region 36 and the back waist region 38. In order for the waistband 122 to receive and maintain tension created by the fastening components, when present, it may be desirable that a portion of the waistband 122 is longitudinally aligned with at least a portion of the fastening system 179. In other words a laterally extending line drawn through the fastener 175 and parallel to the lateral centerline will extend through at least a portion of the elastomeric waistband 122. Alternatively, the tension forces can be transmitted through the waistband 122 as long as a majority or the entirety of the waistband 122 is positioned longitudinally between a laterally extending line from the longitudinally distal edge of one side flap/panel, e.g. side flap longitudinally distal edge 538, side panel longitudinally distal edge 338 to the longitudinally distal edge of the laterally opposing side flap/panel, e.g. side flap longitudinally distal edge 538, side panel longitudinally distal edge 338 and a laterally extending line from the proximal edge of one side flap/panel, e.g. side flap proximal edge 536, side panel proximal edge 336 to the proximal edge of the laterally opposing side flap/panel, e.g. side flap proximal edge 536, side panel proximal edge 336. When the elastomeric waistband 122 is present in only one waist region, e.g. back waist region 38, it may be desirable that the waistband 122 be disposed laterally inward from the waist end edge, e.g. back waist end edge 138, of the article 100. If the waistband 122 is disposed in both waist regions, front waist region 36 and back waist region 38, it may be desirable that the waistband 122 be disposed at or immediately adjacent the front and back waist end edges 136 and 138 of the article 100. The waistband 122 may have a longitudinal length as measured parallel to the longitudinal axis 42. The waistband 122 may be spaced laterally inward from the waist end edge, e.g. back waist end edge 138, a distance that is less than the longitudinal length of the waistband 122. Alternatively, the waistband 122 may be disposed at or adjacent the waist end edge 236 or 238 of the chassis 200 The waistband 122 may be disposed between the side flaps/panels or may overlap a portion of the side flaps/panels to ensure transmission of the tensioning forces generated by the fastener and/or the side flap/panel.

The waistband 122 may be disposed between the topsheet 124 and backsheet 125 of the chassis 200 or may be disposed on the interior surface 202 of the chassis 200. The waistband 122 may be disposed in the same waist region as two elastomeric side flaps/panels or alternatively may be disposed in the same waist region as two non-elastomeric ears 540. In embodiments comprising an elastomeric waistband 122 and non-stretch ears 540, the waistband 122 provides the primary tension along the waist region of the article. The waistband 122 may be disposed wholly between the ears 540 of the article as illustrated in FIG. 41 or a portion of the waistband 122 may be disposed in an overlapping relationship with the ears 540.

The article 100 may comprise an elasticized waistband 122. The elasticized waistband 122 may provide improved fit and containment and may be configured to elastically expand and contract laterally to dynamically fit a wearer's waist. The elasticized waistband 122 may extend longitudinally inwardly from the waist edge of the absorbent article 100 toward the end edges 26 or 28 of the absorbent core 128. In one embodiment, the absorbent article 100 may have two elasticized waistbands 122, one positioned in the back waist region 38 and one positioned in the front waist region 36, although other pant embodiments may be constructed with a single elasticized waistband 122. The elasticized waistband 122 may be constructed in a number of different configurations including those described in U.S. Pat. Nos. 4,515,595 and 5,151,092.

The waistband 122 may comprise a nonwoven layer, an elastomeric layer or combinations thereof. The elastomeric layer may be formed of elastic strands, elastic scrims, elastic films elastic ribbons or elastic nonwovens.

In one embodiment, the elasticized waistbands 122 may comprise materials that have been “prestrained” or “mechanically prestrained” (i.e., subjected to some degree of localized pattern mechanical stretching to permanently elongate the material). The materials may be prestrained using suitable deep embossing techniques. In other embodiments, the materials may be prestrained by directing the material through an incremental mechanical stretching system as described in U.S. Pat. No. 5,330,458. The materials may then be allowed to return to their substantially untensioned condition, thus forming a zero strain stretch material that is extensible, at least up to the point of initial stretching. Examples of zero strain materials are disclosed in U.S. Pat. Nos. 2,075,189, 3,025,199, 4,107,364, 4,209,563, 4,834,741, and 5,151,092.

The waistband 122 may be substantially or entirely disposed between the longitudinally distal laterally extending core edges 26 and 28 and the corresponding waist edge 236 and 238 of the chassis 200 in one or both of the front waist region 36 and back waist region 38.

In certain embodiments of a side panel pant 300, the pant comprises a pair of longitudinally opposing discrete waistbands 122 disposed at or immediately adjacent the longitudinally opposing front and back waist end edges 136 and 138 of the pant.

In embodiments of a taped diaper the diaper comprises a discrete waistband 122 disposed in at least one or both of the front and back waist regions 36 and 38, wherein the waistband 122 is spaced longitudinally inward from the respective front and back waist end edges 136 and 138 of the article 100 at least about 10 mm or at least about 20 mm. The distance the waistband 122 is spaced from the front and/or back waist edge 136 and 138 of the article 100 is typically less than the longitudinal length of the waistband 122.

In either of the articles above, a portion of the elastomeric waistband 122 may be disposed on an interior surface of one or more of the center chassis 200, the topsheet 124, the absorbent core 128 and the inner leg cuffs 150 in one or both of the waist regions of the absorbent article 100. Furthermore, a portion of the elastomeric waistband 122 may be disposed in an overlapping configuration with at least a portion of one or both of the laterally opposing panels/flaps/ears, e.g. elastomeric side panels 330, side flaps 530 and ears 540. Alternatively, a portion of the waistband 122 may be disposed between the topsheet 124 and the backsheet 125 in one or both of the waist regions.

In certain embodiments the waistband 122 may be substantially rectangular. In alternative embodiments, the waistband 122 may have one or more edges that are arcuate as shown in FIG. 47.

### Side Panels

The side panels 330 may be discrete from or integral with the chassis 200. A discrete side panel is formed as separate element, which is joined to the chassis 200. In some embodiments, this includes a plurality of side panels, e.g. 2 or 4 being joined to the side edges 237 of the chassis 200 in the front and/or rear waist regions 36 and 38. Discrete side panels may be formed of one or more layers. The layers of the side panels may include woven materials, nonwovens, films, elastomeric films, foams, elastomeric strands, elastomeric ribbons, elastomeric scrims or combinations thereof. In some embodiments a waistband 122 may be placed across a portion of the chassis 200 and may overlap a portion of the side panels 330 to create a multi-piece continuous belt-like structure. The waistband 122 may be formed of one or more layers. The layers of the waistband 122 may include woven materials, nonwovens, films, elastomeric films, foams, elastomeric strands, elastomeric scrims, elastomeric ribbons or combinations thereof.

In certain embodiments, the absorbent article 100 may comprise a first discrete elastomeric side panel 330 extending outwardly from a first side edge 237 of the chassis 200 in the first waist region and a laterally opposing second discrete elastomeric side panel 330 extending outwardly from a laterally opposing second side edge 237 of the chassis 200 in the first waist region and wherein the longitudinally distal edge 236 or 238 of the chassis in the first waist region and the longitudinally distal edge 236 or 238 of the laterally opposing side panels 330 in the first waist region form a portion of the waist opening 190.

In an alternative embodiment the absorbent article 100 may comprise a pair of discrete elastomeric side panels 330 disposed in a first waist region and a pair of discrete non-elastomeric ears 540 disposed in a second waist region.

In another embodiment the article may comprise a pair of discrete elastomeric side panels 330 disposed in a first waist region and a pair of integral non-elastomeric ears 540 in a second waist region wherein the integral non-elastomeric ears 540 may be formed in part by one or more of the backsheet nonwoven 127, backsheet film 126, cuff material, landing zone material and topsheet 124.

In certain embodiments the discrete elastomeric side panels 330 may overlap the side edges 237 of the chassis 200 and may be disposed on the innermost surface 202 of the chassis 200 or alternatively to the outermost surface 204 of the chassis 200. Alternatively, the elastomeric side panels 330 may overlap a portion of the chassis 200 and may be disposed between layers of the chassis 200 for example between the backsheet 125 and the outer leg cuff 140, or between the backsheet 125 and the topsheet 124 or may be disposed on either the interior surface 202 or exterior surface 204 of the chasis 200. The side panels 330 may be bonded to the chassis 200 adhesively, mechanically, thermally or by combinations thereof.

The elastomeric side panels 330 may comprise an apertured elastomeric film material, elastic strands, elastomeric scrims materials, elastomeric nonwovens elastic ribbons, foams and combinations thereof. The elastomeric side panels 330 comprising elastic strands may comprise at least two or more strands having a spacing between the two or more strands of from 2 mm to about 20 mm. The elastic strands may also have a dtex of between 155 and 1880 and may have an extension ratio of between 125% and 400%. The elastomeric material of the side panel 330 may be the same color as the nonwoven of the side panel 330 or may be of a different color so the elastomeric material is visible in the elastomeric side panel 330. The elastomeric side panels 330 may also comprise a nonwoven layer having a basis weight between 6 and 36 grams per square meter.

The side panels 330 may be substantially rectangular or may have a shape, i.e. the side panels 330 are substantially non-rectangular. In addition to shape the side panels 330 may also comprise two or more visually distinct textural regions disposed on the side panel 330. Finally one or more of the side panels 330 of the side panel pant 300 may comprise a side panel graphic 399 and the chassis 200 may comprise a chassis graphic 299 wherein the side panel graphic and the chassis graphics are substantially aligned.

The side panels 330 may be discrete elements attached to the chassis 200 at or adjacent the side edges 237 of the chassis 200. Alternatively the side panels 330 may be integral with the chassis 200, i.e. formed in part by one or more of the backsheet nonwoven, backsheet film, outer cuff material, topsheet and core wrap.

### Belt Flaps

The belt flaps 430 disposed on a belt flap pant 400 may comprise an inner belt layer 432 and an outer belt layer 434 layer and an elastomeric material disposed there between. The inner and outer nonwoven layers 432 and 434 may be joined using adhesives, heat bonds, pressure bonds or thermoplastic bonds. Various suitable belt flap configurations can be found in U.S. Pub. No. 2013-0211363.

A belt flap pant 400 may comprise a first elastomeric belt flap 430 extending outwardly from a first end edge, e.g., back end edge 238, of the chassis 200 and a longitudinally opposing second elastomeric belt flap 430 extending outwardly from a longitudinally opposing second end edge, e.g. front end edge 236 of the chassis 200. The longitudinally distal edge 437 of the first elastomeric belt flap 430 and the longitudinally distal edge 437 of the second elastomeric belt flap 430 form a portion of a waist opening 190.

In certain embodiments, the absorbent article 100 may comprise a first belt flap 430 disposed in a first waist region, e.g. the back waist region 38, and a second belt flap 430 disposed in a longitudinally opposing second waist region, e.g. the front waist region 36, wherein one of the belt flaps has a greater longitudinal length than the longitudinally opposing belt flap as measured along the side edge 437 of the belt flap 430 at or adjacent the side seam 170.

In one embodiment an absorbent belt flap pant 400 may comprise a first elastomeric belt flap 430 and a second elastomeric belt flap 430 that are discrete from one another. Each of the belt flaps may comprise an inner belt layer 432 and an outer belt layer 434 wherein the inner belt layer 432 on the first belt flap 430 is separated by a gap from the inner belt layer 432 on the second belt flap 430 and the outer belt layer 434 on the first belt flap 430 is separated by a gap from the outer belt layer 434 on the second belt flap 430.

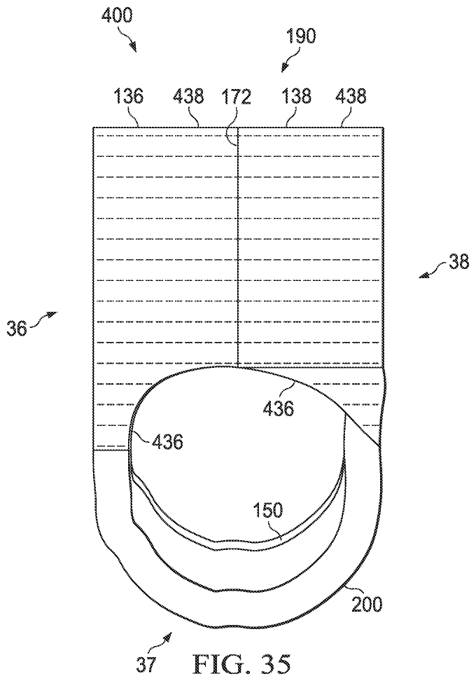

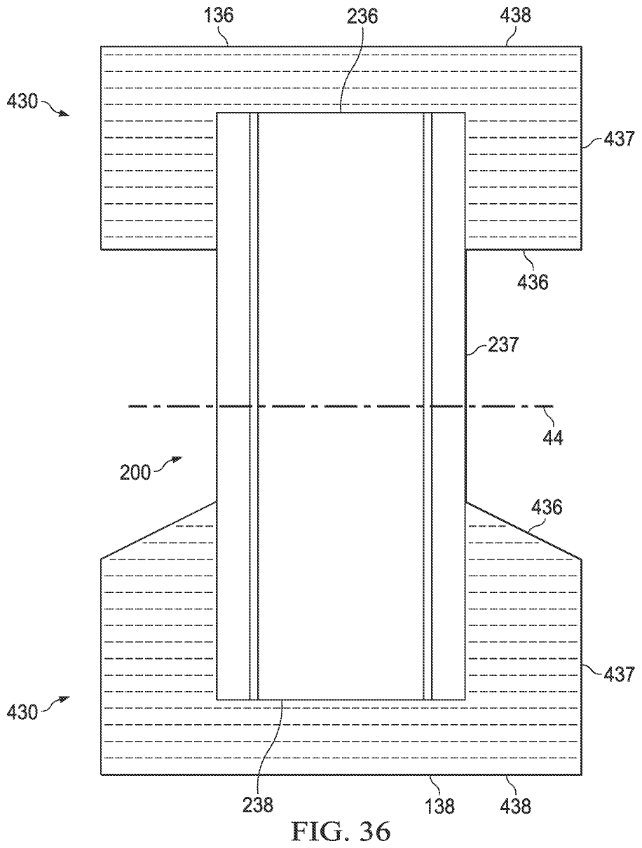

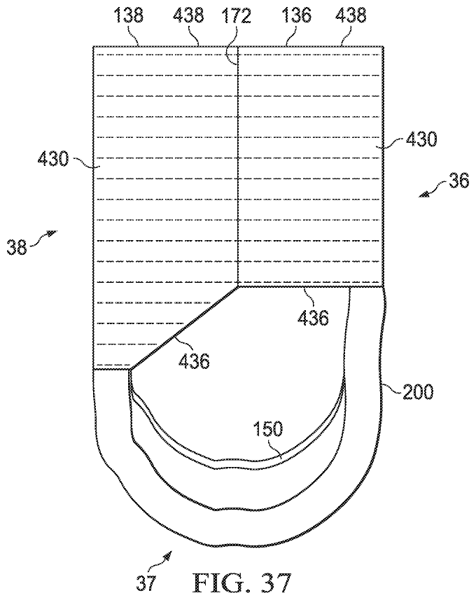

In an alternative embodiment an absorbent belt flap pant 400 may comprise a first elastomeric belt flap 430 in a first waist region and a second elastomeric belt flap 430 in a second waist region. Each of the belt flaps 430 may comprise an inner belt layer 432 and an outer belt layer 434 wherein one or both of the inner belt layer 432 and the outer belt layer 434 form a common belt layer extending from a first waist region to a second waist region forming a portion of the first belt flap 430 and a portion of the second belt flap 430. In certain embodiments the common layer may extend from a first waist edge, e.g. the front waist edge 136 to a longitudinally opposing second waist edge, e.g. back waist edge 138 of the article 100. Alternative embodiments of the belt flap 430 may include slits, holes or perforations providing increased breathability, softness and garment like texture. Underwear-like appearance can be enhanced by substantially aligning the waist edge, i.e. longitudinally distal edge 438 and/or leg edge, i.e. longitudinally proximal edge 436 of the first and second belt flaps 430 of the belt flap pant 400 providing a smooth waist opening 190 and smooth leg openings 192 as illustrated in FIG. 35.

In certain embodiments, one or both of the belt flaps 430 on the belt flap pant 400 may comprise belt flap graphics 499. The belt flap graphics 499 may extend substantially around the entire circumference of the belt flap pant 400. The belt flap graphic 499 may extend around the circumference at least about 180 degrees or at least about 225 degrees or at least about 270 degrees. The belt flap graphics 499 (or color scheme) on the belt flap 430 may be substantially aligned with chassis graphics 299 disposed on the chassis 200.

### Side Flaps

Taped diaper absorbent articles 500 may comprise a first elastomeric side flap 530 extending outwardly from a first side edge 237 of the chassis 200 and a laterally opposing second elastomeric side flap 530 extending outwardly from a laterally opposing second side edge 237 of the chassis 200. The taped diaper 500 may comprise a chassis 200 having a longitudinally distal edge, e.g. back end edge 238, disposed in a first waist region, e.g. back waist region 38. The longitudinally distal edge of the chassis 200 and the longitudinally distal edges 538 of the laterally opposing first elastomeric side flap 530 and the second elastomeric side flap 530 form a portion of a waist edge, e.g. back waist edge 138, of the taped diaper 500.

The taped diaper 500 may also comprise non-elastomeric ears 540 that may be combined with an elastomeric waistband 122 to create an elasticized waist opening 190. The ears 540 may be rectangular or may be shaped, substantially non-rectangular. The ears may be in one or both of the waist regions and may be discrete and attached to the chassis 200 along the side edges 237 or alternatively they may be integral and formed in part by a portion of one or more of the backsheet 125, topsheet 124, leg cuff, or landing zone. In certain executions, the side flaps 530 or ears 540 of the taped diaper 500 may comprise at least two visually distinct textured zones.

In certain embodiments one or more of the side flaps 530 of the taped diaper 500 may comprise a side flap graphic 599 disposed thereon. In some embodiments one or more of the side flaps 530 comprise a side flap graphic 599 and the chassis 200 comprises a chassis graphic 299. In certain executions, the side flap graphic 599 disposed on the side flap 530 and the chassis graphic 299 disposed on the chassis 200 may be substantially aligned.

The side flaps 530 may overlap a portion of the chassis 200 and may be disposed between layers of the chassis 200 for example between the backsheet 125 and the outer leg cuff 140, or between the backsheet 125 and the topsheet 124 or may be disposed on either the interior surface 202 or exterior surface 204 of the chassis 200. The side flaps 530 may be bonded to the chassis 200 adhesively, mechanically, thermally or by combinations thereof.

The side flaps 530 may be discrete elements attached to the chassis 200 at or adjacent the side edges 237 of the chassis 200. Alternatively the side flaps 530 may be integral with the chassis 200, i.e. formed in part by one or more of the backsheet nonwoven, backsheet film, outer cuff material, topsheet and core wrap. The side flaps may be elastomeric or may be non-elastomeric.

### Fastening Systems

The absorbent article 100 may also include a fastening system 179. When fastened, the fastening system 179 interconnects the front waist region 36 and the rear waist region 38 resulting in a waist circumference that may encircle the wearer during wear of the absorbent article 100. This may be accomplished by connecting the side panels 330 in one waist region with the side panels 330 in the longitudinally opposing waist region (e.g. a refastenable seam in a side panel pant 300) or by connecting side flaps 530 in one waist region with the chassis 200 in the longitudinally opposing waist region (e.g. a closure in a taped diaper 500). The fastening system 179 may comprises a fastener 175 such as tape tabs, hook and loop fastening components, interlocking fasteners such as tabs & slots, buckles, buttons, snaps, and/or hermaphroditic fastening components, although any other known fastening means are generally acceptable. The fasteners 175 may releasably engage with a mating fastener 178, which may comprise a woven or nonwoven substrate. In certain embodiments the fastening system 179 may comprise a fastener 175 joined to a fastener tab which is in turn may be joined to the side panel 330 or side flap 530. In other embodiments the fastener 175 is joined directly to the side panel 330 or side flap 530 of the article 100. In certain embodiments each side flap 330 may be fastened to a portion of the chassis 200 by a primary fastener 175 disposed on the side flap 330 or on a fastener tab connected to the side flap 330 and by a mating fastener 178 disposed on the chassis 200 designed to engage with the fastener 175 on the side flap 330. In some embodiments the fastener 175 and fastener tab may be substantially rectangular. In alternative embodiments the fastener 175 and/or fastener tab may be shaped or rounded. Some exemplary surface fastening systems 179 are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098. The fastening system 179 including a disposal tape may also provide a means for holding the article in a disposal configuration as disclosed in U.S. Pat. No. 4,963,140. The fastening system 179 may also include primary fasteners 175 and mating fasteners 178, as disclosed in U.S. Pat. No. 4,699,622. The fastening system 179 may be constructed to reduce shifting of overlapped portions or to improve fit as disclosed in U.S. Pat. Nos. 5,242,436; 5,499,978; 5,507,736; and 5,591,152.

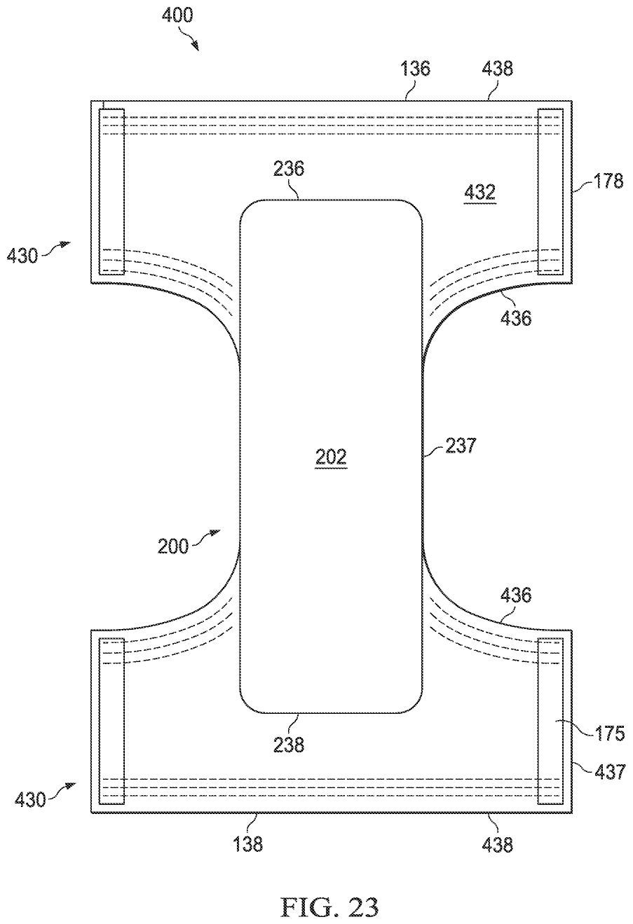

Side panel pants 300 comprising a fastening system 179 forming a refastenable side seam 174 may comprise a primary fastener 175 disposed on an interior surface of one or more of the side panels 330 in a first waist region and a mating fastener 178 disposed on an exterior surface of one or more of the side panels 330 disposed in a second waist region. Alternatively, the side panel pants 300 comprising a fastening system 179 forming a refastenable side seam 174 may comprise a primary fastener 175 disposed on an exterior surface of one or more of the side panels 330 in a first waist region and a mating fastener 178 disposed on an interior surface of one or more of the side panels 330 disposed in a second waist region. The primary fasteners 175 may be attached to the surface of the side panels 330 in a first waist region and the mating fastener 178 may be attached to or form a portion of the side panels 330 in a second waist region as illustrated in FIGS. 22 and 23 as well as FIGS. 1-3.

Belt flap pants 400 comprising a fastening system 179 forming a refastenable side seam 174 may comprise a fastener 175 disposed on an interior surface of the belt flap 430 in a first waist region and a mating fastener disposed on an interior surface of the longitudinally opposing belt flap 430 in a second waist region.

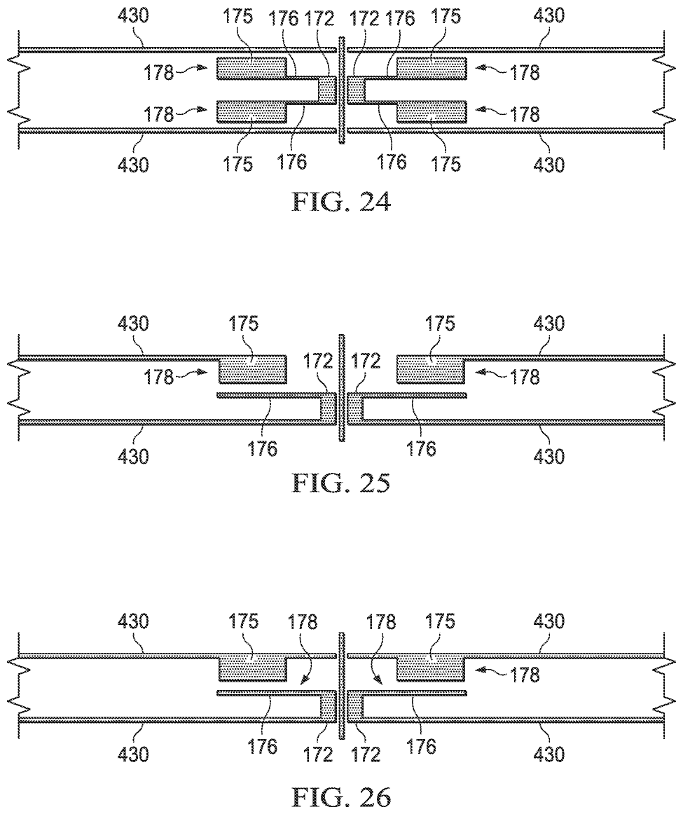

In certain embodiments shown in FIGS. 22-29 the belt flap pant 400 may comprise a fastening system 179 having a primary fastener 175, a fastening tab 176 and a mating fastener 178. In FIG. 24 the fastening system 179 is disposed between the front and back belt flaps 430. The mating fastener 178 forms at least a portion of the body facing surface of the belt flaps 430 and the primary fastener 175 is disposed on a fastener tab 176. The primary fastener 175 engages with the mating fastener 178 forming a portion of the interior surface of the belt flaps 430. The fastening system 179 forms dual refastenable seams 174, one refastenable seam 174 disposed in the front waist region 36 and one refastenable seam 174 in the back waist region 38. The fastener tabs are joined to each other by a permanent seam 172 to complete the waist opening 190. The refastenable seam is opened by disengaging the primary fastener 175 from the mating fastener 178 in one or both of the front and back waist regions 36 and 38. The refastenable seam may be reclosed by reengaging the primary fastener 175 and the mating fastener 178.

In FIG. 25 the fastening system 179 is disposed between the front and back belt flaps 430. The mating fastener 178 forms a portion of the body facing surface of the front belt flap 430 and the primary fastener 175 is disposed in an engaged fashion with the body facing surface of the front belt flap 430. The fastening system 179 also comprises a fastening tab 176 that is permanently seamed to the back belt flap 430 at or adjacent the side edge 437. The fastener 175 and the fastening tab 176 are joined to each other via an adhesive, cohesive, or other means known in the art to complete the waist opening 190. The refastenable seam is opened by disengaging the primary fastener 175 from the mating fastener 178. The refastenable seam may be reclosed by reengaging the primary fastener 175 and the mating fastener 178.

In FIG. 26 the fastening system 179 is disposed between the front and back belt flaps 430. The primary fastener 175 is permanently joined to the front belt flap 430 adjacent the side edge 437 of the belt flap 430. The mating fastener 178 forms at least a portion of the fastening tab 176. The fastening tab 176 is permanently seamed to the back belt flap 430 at or adjacent the side edge 437. The fastener 175 disposed on the front belt flap 430 is refastenably engaged with the fastening tab 176 to complete the waist opening 190. The refastenable seam is opened by disengaging the primary fastener 175 from the fastening tab 176 which is formed at least in part by the mating fastener 178. The refastenable seam may be reclosed by reengaging the primary fastener 175 and the fastening tab 176.

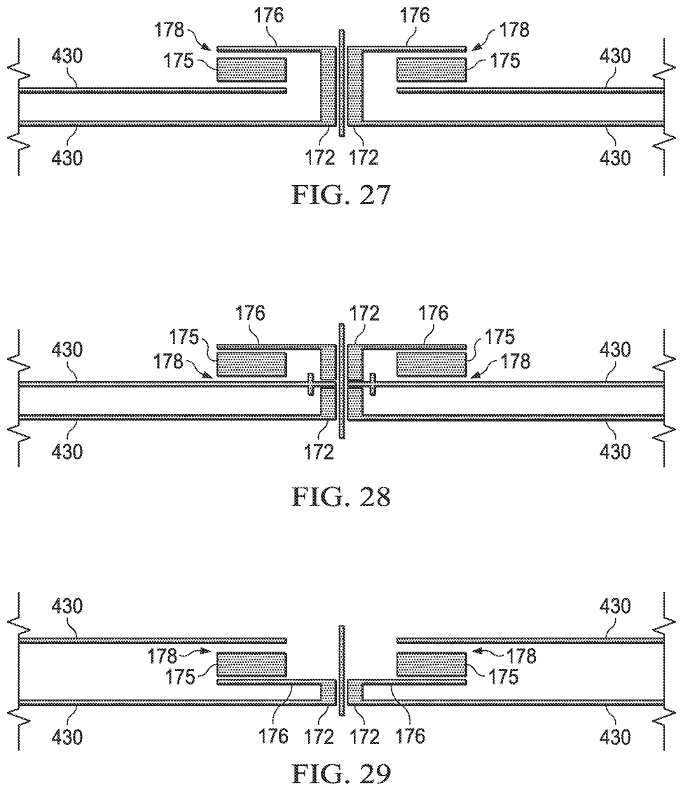

In FIG. 27 the fastening system 179 is disposed on the exterior surface of the front belt flap 430. The primary fastener 175 is permanently joined to the front belt flap 430 adjacent the side edge 437 of the belt flap 430. The mating fastener 178 forms at least a portion of the fastening tab 176. The fastening tab 176 is permanently seamed to the back belt flap 430 at or adjacent the side edge 437. The fastener 175 disposed on the front belt flap 430 is refastenably engaged with the fastening tab 176 to complete the waist opening 190. The refastenable seam is opened by disengaging the primary fastener 175 from the fastening tab 176 which is formed at least in part by the mating fastener 178. The refastenable seam may be reclosed by reengaging the primary fastener 175 and the fastening tab 176.

In FIG. 28 the fastening system 179 is disposed on the exterior surface of the front belt flap 430. The primary fastener 175 is permanently joined to the fastening tab 176. The mating fastener 178 forms at least a portion of exterior surface of the front belt flap 430. The fastening tab 176 is permanently seamed to the front belt flap 430 and the back belt flap 430 at or adjacent the side edge 437. A perforation is disposed between the primary fastener 175 and the permanent side seam 172. The fastener 175 disposed on the fastening tab 176 is refastenably engaged with the mating fastener 178 to complete the waist opening 190. The refastenable seam is opened by disengaging the primary fastener 175 from the mating fastener 178 which forms at least a portion of the front belt flap 430 subsequently breaking the perforation disposed adjacent the permanent side seam 172 and opening the pant. The refastenable seam may be reclosed by engaging the primary fastener 175 to the exterior surface of the front belt flap 430.

In FIG. 29 the fastening system 179 is disposed between the front and back belt flaps 430. The mating fastener 178 forms a portion of the body facing surface of the front belt flap 430 and the primary fastener 175 is disposed on a fastening tab 176. The fastening tab 176 is permanently seamed to the back belt flap 430 at or adjacent the side edge 437. The primary fastener 175 is refastenable engaged with the body facing surface of the front belt, i.e. mating fastener adjacent the side edge 437 of the belt flap 430 to complete the waist opening 190. The refastenable seam is opened by disengaging the primary fastener 175 from the mating fastener 178. The refastenable seam may be reclosed by reengaging the primary fastener 175 and the mating fastener 178.

For the belt flap refastenable executions, the references to front belt flap 430 and back belt flap 430 should be considered to be interchangeable such that a fastener 175 that is described above in the front waist region 36 may be disposed in the back waist region 38 in a similar structural configuration.

### Identical or Substantially Identical Chassis

As disclosed in U.S. Pub. No. 2013-0211355, it may be desirable to offer an array of packages for fitting wearers having different needs, but comprising identical or substantially identical chassis 200. For instance, an array may comprise a first package comprising a first absorbent article 100 and a second package may comprise a second absorbent article 100, where the first and second packages comprise articles having identical or substantially identical chassis 200 as described in U.S. Pub. No. 2013-0211355. More particularly, the first package may comprise a first chassis 200 and the second package may comprise a second chassis 200, where each of the first and second chassis comprise the same dimensions of one or more of: core width at the lateral axis 44, core width at one of the front or rear core edge 26 and 28, a distance from a left outer cuff distal edge 142 to a right outer cuff distal edge 142, a distance from a left inner cuff proximal edge 152 to a left outer cuff distal edge 142, a distance from a left inner cuff proximal edge 152 to a right inner cuff proximal edge 152, a distance from a left inner cuff proximal edge 152 to a left outer cuff distal edge 142, a free height of the inner cuff, inner cuff hem fold width, inner cuff elastics length, outer cuff elastics length, core length, chassis length and backsheet width.

Further, each of the first and second chassis 200 may comprise identical chemical compositions of one or more of a topsheet 124, backsheet film 126, backsheet nonwoven 127, core super absorbent polymers, core pulp, core nonwoven, core tissue, leg cuff film, leg cuff nonwoven, super absorbent polymer adhesive, core nonwoven adhesive, leg cuff elastic adhesive, and backsheet nonwoven/film adhesive.

And, each of the first and second chassis 200 may comprise the same basis weight of one or more of the topsheet 124, backsheet film 126, backsheet nonwoven 127, core super absorbent polymers, core pulp, leg cuff nonwoven, leg cuff film, super absorbent polymer adhesive, leg cuff adhesive, and backsheet nonwoven/film adhesive.

And, each of the first and second chassis 200 may comprise compositionally identical core super absorbent polymers. The first and second chassis 200 may have identical component cross sectional order and disposition in at least one of the front waist region 36, back waist region 38, and crotch region 37. The inner leg cuffs 150 of the first and second chassis 200 may be composed of the compositionally identical materials.

And, the core adhesives of the first and second chassis 200 may be the same adhesive(s). The first and second chassis 200 may comprise core super absorbent polymers that are in the same chemical class and subclass.

And, each of the first and second chassis 200 may comprise first and second wetness indicators, respectively, and wherein the first and second wetness indicators are compositionally identical.

Further, the inner leg cuffs 150 of the first and second chassis 200 may have identical component cross sectional order and disposition in at least one of the front waist region 36, back waist region 38, and crotch region 37. The distance from the left outer cuff distal edge 142 to a right outer cuff distal edge 142 may the same. The distance from the left inner cuff proximal edge 152 to left outer cuff distal edge 142 may be the same. The distance from the left inner cuff proximal edge 152 to the right inner cuff proximal edge 152 is the same. The lengths of the inner and outer cuffs 150 and 140 are the same.

In some embodiments, different product offerings in an array may have identical or substantially identical chassis 200 as the side panels 330, side flaps 530 or belt flaps 430 may be used to distinguish the absorbent article forms one from another. For example, first and second absorbent articles may have identical chassis (compositionally, dimensionally, cross-sectionally), but the first article 100 may have a different length due to disposition of the belt flaps. As a second example, first and second absorbent articles 100 may have identical chassis (compositionally, dimensionally, cross-sectionally), but the first article may have a different length and/or width due to the size of the belt flaps.

In some embodiments, first and second absorbent articles 100 may have identical chassis compositionally, but not dimensionally, and not cross-sectionally. In some embodiments, first and second absorbent articles 100 may have identical chassis dimensionally, but not compositionally, and not cross-sectionally. In some embodiments, first and second absorbent articles 100 may have identical chassis cross-sectionally, but not dimensionally, and not compositionally. In still other embodiments, first and second absorbent articles 100 may have two, but not three of (1) compositionally, (2) dimensionally, and (3) cross-sectionally identical chassis.

It should be noted that for all of the above statements relating to identical or substantially identical chassis that a third package comprising a third article 100 may be added. It should also be noted that the structure and/or form of the first article 100, second article 100 and third article 100 when present can all be different in other words the first article 100 may be an open form, taped diaper 500, the second article may be a closed form, side panel refastenable pant 300, and the third article 100 may be a closed form, belt flap pant 400.

### Graphics

There is a desire to make disposable absorbent articles 100 look more like underwear. This desire is driven by the desire to address the psychological and emotional development needs of the wearer. There are several factors that can affect whether an absorbent article 100 is perceived as underwear-like. These factors include, but are not limited to, the noticeability of seams 170, the existence of graphics on a larger percentage of the viewable surfaces of the absorbent article 100, the appearance of waist and leg features and graphics flowing from or over two or more absorbent article components.

There are, however, many obstacles to designing and executing an absorbent article 100 that is underwear-like. One is that absorbent articles are a compilation of separate article components and as new disposable absorbent article technologies are developed, they may result in the need for incorporation of additional, separate, article components. Because of the manner in which absorbent article components are incorporated, even the viewable surfaces of the article may have seams or areas of overlap or connection. Thus, it is challenging to place graphics on these individual components and to line them up such that the multi-component construction appears to be an integrated structure wherein the seams are de-emphasized and process variations are masked.

Further, depending on the type of article construction, it is challenging to maintain the uniform appearance of graphics that may be printed on more than one component. For example, a component with a particular graphic printed on it may eventually be incorporated into the article at a different depth than an adjacent component that also has a graphic printed on it. Depending on whether particular portions of a graphic are printed on an outermost layer, for example, or, if not, how opaque the layers may be through which the various portions of the graphic may be viewed, can have an impact on an entire article graphic that is meant to look uniform and consistent in that the various portions may have measureable differences in appearance when printed on different components or at different depths relative to the outer surface. For these and other reasons, it is challenging to create an absorbent article that comprises mainstream technologies and also that comprises graphics on multiple absorbent article components, such that the graphics flow from one absorbent article component to another article component in a manner that deemphasizes transitions from one component to another and creates a holistic underwear-like appearance.

It is a desire of the present disclosure to provide absorbent articles 100 that look like underwear, while, at the same time, incorporating multiple absorbent article components that offer the benefits and functionality of the latest technologies (e.g., high stretch components integrated with low/no stretch components, highly breathable components, etc.). More specifically, it is a desire of the present disclosure to provide absorbent articles 100 comprising graphics disposed on or spanning multiple viewable absorbent article components while creating a uniform appearance.

As shown in FIGS. 4, 5, 8 and 9, a disposable absorbent article 100 can have 2 or 4 elastomeric side flaps 530 or side panels 330 that overlap with a portion of the chassis 200 or an elastomeric belt flap 430 disposed in one or both of the waist regions that overlap with a portion of the chassis 200. In certain embodiments the side flaps 530 or side panels 330 may comprise a first graphic element and the chassis 200 may comprise a second graphic element wherein the graphic elements are designed to work in concert with each other to create a composite graphic element. In one embodiment a first elastomeric side flap 530 or side panel 330 comprises a first graphic extending from a side edge of the side flap or side panel 537 and 337 respectively toward the longitudinal axis 42, a second elastomeric side flap 530 or side panel 330 comprises a second graphic extending from a side edge of the side flap or side panel 537 and 337 respectively toward the longitudinal axis 42 and the center chassis 200 comprises a third graphic extending from a first side edge 237 of the chassis to the opposing side edge 237 of the chassis wherein the first, second and third graphic are substantially aligned. In general a composite graphic is formed when two or more separate graphics are substantially aligned to form a larger continuous graphic.

As shown in FIGS. 15, 16 and 21, disposable absorbent articles comprising one or more belt flaps 430 a first graphic may be printed on a surface of one of the layers of one of the first belt flap or second belt flaps 430, a second graphic may be printed on a surface of one of the group consisting of a nonwoven layer of the first belt flap 430 or second belt flap 430, a layer of the chassis 200, a backsheet 125, and an outer cover, e.g. backsheet nonwoven 127. In some embodiments, additional graphics may be printed on other layers of the article, for example, any of the layers of the belt flaps 430, the elastomeric material, the chassis 200, or of the backsheet 125 or outer cover. In one embodiment a first elastomeric belt flap 430 comprises a first graphic element and the chassis 200 comprises a second graphic element wherein the first and second graphic elements are substantially aligned.

For embodiments comprising a belt flap 430, it is desirable that the arrangement of graphic elements cover greater than about 30% of the surface area of the belt flap 430 or greater than about 40% or greater than about 50%. In some embodiments the graphic elements may cover greater than 75% of the surface area of the belt flap 430. Additionally the arrangement of graphics may when viewed from the outside appear to comprise at least 3 colors or at least 5 colors or greater than 7 colors. To provide a very underwear-like appearance the arrangement of graphic elements may be disposed within 30 mm of the waist edge 136 and 138 in one or both of the waist regions, or within 20 mm or within 10 mm of the leg opening edge. In certain embodiments the graphics elements may be disposed at or immediately adjacent the waist edge of one or both of the belt flaps 430. In addition it may be desirable that the arrangement of graphic elements may also be disposed within about 10 mm of a leg edge of the belt flap 430.

For embodiments comprising discrete side panels 330 or side flaps 530, it is desirable that the arrangement of graphic elements cover greater than about 10% of the surface area of the side panel 330 or side flap 530 or greater than about 25% or greater than about 50%. In some embodiments the graphic elements may cover greater than 75% of the surface area of the discrete side panel 330 or side flap 530. Additionally the arrangement of graphics may when viewed from the outside appear to comprise at least 3 colors or at least 5 colors or greater than 7 colors. To provide a very underwear-like appearance the arrangement of graphic elements may be disposed within 30 mm of the waist edge in one or both of the waist regions or within 20 mm or within 10 mm of the leg opening edge. In certain embodiments the graphics elements may be disposed at or immediately adjacent the waist edge of the side panels 330 or side flaps 530. In addition it may be desirable that the arrangement of graphic elements may also be disposed within about 10 mm of a leg opening edge of the side panel 330 or side flap 530.

### Sensors

The absorbent articles of the present disclosure may comprise a sensor system 700 for monitoring changes within the absorbent article 100. The sensor system 700 may be discrete from or integral with the absorbent article 100. The absorbent article 100 may comprise sensors that can sense various aspects of the absorbent article 100 associated with insults of bodily exudates such as urine and/or BM (e.g., the sensor system 700 may sense variations in temperature, humidity, presence of ammonia or urea, various vapor components of the exudates (urine and feces), changes in moisture vapor transmission through the absorbent articles garment-facing layer, changes in translucence of the garment-facing layer, color changes through the garment-facing layer, etc.). Additionally, the sensor system 700 my sense components of urine, such as ammonia or urea and/or byproducts resulting from reactions of these components with the absorbent article 100. The sensor system 700 may sense byproducts that are produced when urine mixes with other components of the absorbent article 100 (e.g., adhesives, agm, etc.). The components or byproducts being sensed may be present as vapors that may pass through the garment-facing layer. It may also be desirable to place reactants in the diaper that change state (e.g. color, temperature, etc.) or create a measurable byproduct when mixed with urine or BM. The sensor system 700 may also sense changes in pH, pressure, odor, the presence of gas, blood, a chemical marker or a biological marker or combinations thereof.

The sensor system 700 or portions thereof may be integrated with the absorbent article 100 with hook and loops fasteners, adhesives, thermal bonds, mating fasteners like snaps or buttons, or may be disposed in pockets, recesses or void spaces built into the absorbent article 100, or combinations thereof. Many of these integration means enable removal of and/or attachment of the sensor system 700 or portions thereof from or to the absorbent article 100. The absorbent article 100 may further comprise graphics for the purpose of properly locating the sensor system 700 or portions thereof. In addition, in cases where an auxiliary article is present, the auxiliary article may be joined to the absorbent article 100 by similar integration means. Regarding pockets, it may be desirable to form a pocket within or adjacent to the wearer-facing layer or garment-facing layer of the article. In some embodiments, a pocket may be formed by joining an additional material (e.g., a nonwoven strip) to the interior or exterior surface of the garment-facing layer. When joined to the interior surface of the garment facing layer, it may be desirable to position an open edge (to be the pocket opening) of the sheet to be coterminous or adjacent to an edge of the waist opening 190 such that there is no need to make a cut in the garment facing layer for inserting the sensor system 700 or portions thereof into the pocket opening.

As used in this application, the term “sensor system” refers not only to the elements responsible for detecting a stimulus and/or change in status of the article and signaling such detection, but also may include the housing or carrier layer or substrate around such element(s). A “sensor system” may include a carrier layer with multiple elements capable of detecting one or more stimuli; and, the multiple elements may create multiple locations capable of detecting one or more stimuli. The sensor systems 700 of the present disclosure may be formed of multiple components capable of monitoring urine and/or fecal insults. The sensor system may take on a variety of configurations, which are determined by the means in which the presence of urine and/or feces is detected. After detection of urine and/or feces, the sensor system may inform a caregiver and/or a child by generating a notification. The notification may be an auditory signal, an olfactory signal, a tactile signal or a visual signal. It is understood that the sensor system may comprise a device for sending a wireless signal to a remote receiver which may in turn result in an auditory signal, visual signal, tactile signal or other sensory signal and/or combinations thereof.

Various sensors may be used as part of the sensor system, including inductive, capacitive, ultra-sonic, optical, moisture, humidity (e.g., MVTR), pH, biological, chemical, mechanical, temperature, electromagnetic and combinations thereof, as described and illustrated in U.S. Pub. Nos. 2012/0310190 and 2012/0310191.

The sensor system may include one or more transmitters. A transmitter is a device that sends electromagnetic waves carrying messages or signals, for instance, one or more of the sensor elements may comprise a transmitter. Alternatively, a transmitter may be removably fixed to the absorbent article 100 or to an auxiliary article such that it is in contact or in communication with the sensor elements.

### Absorbent Cores

As used herein, the term “absorbent core” refers to the individual component of the absorbent article 100 having the most absorbent capacity and that comprises an absorbent material. The absorbent core 128 may comprise a core wrap or core bag (hereafter “core wrap”) enclosing the absorbent material. The absorbent core 128 may comprise, consist essentially of, or consist of, a core wrap, absorbent material as defined below, and glue enclosed within the core wrap. Pulp or air-felt may also be present within the core wrap and may form a portion of the absorbent material. The absorbent core periphery, which may be the periphery of the core wrap, may define any suitable shape, such as a “T,” “Y,” “hour-glass,” or “dog-bone” shape, for example. An absorbent core periphery having a generally “dog bone” or “hour-glass” shape may taper along its width towards the middle or “crotch” region of the core. In this way, the absorbent core 128 may have a relatively narrow width in an area of the absorbent core 128 intended to be placed in the crotch region of an absorbent article 100.

The absorbent article 100 may comprise an absorbent core 128 (also referred to as an “absorbent member” or “absorbent assembly” or “absorbent structure” or “absorbent composite”) that is disposed between the topsheet 124 and the backsheet 125. In one embodiment, more than one absorbent core 128 or more than one absorbent core layer may be provided in an absorbent article 100, for example. The absorbent core 128 may be any suitable size or shape that is compatible with the absorbent article 100. Example absorbent structures for use as the absorbent core 128 of the present disclosure that have achieved acceptance and commercial success are described in U.S. Pat. Nos. 4,610,678; 4,673,402; 4,888,231; and 4,834,735.

In one embodiment, suitable absorbent cores 128 may comprise cellulosic airfelt material. For instance, such absorbent cores may comprise less than about 40%, 30%, 20%, 10%, 5%, or even 1% of the cellulosic airfelt material as determined by weight. Additionally, such an absorbent core may be primarily comprised of an absorbent gelling material in amounts of at least about 60%, 70%, 80%, 85%, 90%, 95%, or even about 100% as determined by weight. Furthermore, a portion of the absorbent core 128 may comprise a microfiber glue (if applicable). Such absorbent cores, microfiber glues, and absorbent gelling materials are described in U.S. Pat. Nos. 5,599,335; 5,562,646; 5,669,894; 6,790,798; and 7,521,587 and in U.S. Pat. Publ. No. 2004/0158212.

In one embodiment, the core, including multiple layers making up the core system, may be printed and/or embossed as described in U.S. Pat. No. 8,536,401.

In one embodiment, the absorbent core 128 may be separable from the chassis as disclosed in U.S. Pat. Nos. 6,989,006; 7,381,202; 7,175,613; 7,824,386; 7,766,887; and 6,989,005. In such embodiments, the measurements described in this disclosure may be made to the chassis alone or may be made to the chassis in combination with the separable core/absorbent assembly.

“Absorbent material” means a material which has some absorbency property or liquid retaining properties, such as SAP, cellulosic fibers as well as synthetic fibers. Typically, glues used in making absorbent cores have no absorbency properties and are not considered as absorbent material.

One embodiment of an absorbent core 128 of the absorbent article 100 comprises a front core edge 26, a back core edge 28, and two core side edge 27 joining the front core edge 26 and the back core edge 28. The absorbent core 128 may also comprise a generally planar top side and a generally planar bottom side. The front core edge 26 of the core 128 is the edge of the core 128 intended to be placed towards the front waist edge 136 of the absorbent article 100. The core 128 may have a longitudinal axis 42 corresponding substantially to the longitudinal axis 42 of the absorbent article 100. The absorbent material may be distributed in higher amount towards the front core edge 26 than towards the back core edge 28 as more absorbency may be required at the front in particular articles, e.g. articles intended for boys. The absorbent material may have a non-uniform basis weight or a uniform basis weight across any portion of the absorbent core 128. The core wrap may be formed by two nonwoven materials, substrates, laminates, or other materials, which may be at least partially sealed along the sides and ends of the absorbent core 128. The core wrap may be at least partially sealed adjacent its front core edge 26, back core edge 28, and adjacent the two core side edges 27 to prevent absorbent material from leaking out of the absorbent core wrap.

Cores comprising relatively high amount of SAP with various core designs are disclosed in U.S. Pat. No. 5,599,335 (Goldman), EP 1,447,066 (Busam), WO 95/11652 (Tanzer), U.S. Pat. Publ. No. 2008/0312622A1 (Hundorf), and WO 2012/052172 (Van Malderen).

The absorbent material may be one or more continuous layers present within the core wrap. Alternatively, the absorbent material may be comprised of individual pockets or stripes of absorbent material enclosed within the core wrap. In the first case, the absorbent material may be, for example, obtained by the application of a single continuous layer of absorbent material. The continuous layer of absorbent material, in particular of SAP (super absorbent polymer), may also be obtained by combining two or more absorbent layers having discontinuous absorbent material application patterns, wherein the resulting layer is substantially continuously distributed across the absorbent particulate polymer material area, as disclosed in U.S. Pat. Appl. Publ. No. 2008/0312622A1 (Hundorf), for example. The absorbent core 128 may comprise a first absorbent layer and a second absorbent layer. The first absorbent layer may comprise the first material and a first layer of absorbent material, which may be 100% or less of SAP. The second absorbent layer may comprise the second material and a second layer of absorbent material, which may also be 100% or less of SAP. The absorbent core 128 may also comprise a fibrous thermoplastic adhesive material at least partially bonding each layer of absorbent material to its respective material.

The fibrous thermoplastic adhesive material may be at least partially in contact with the absorbent material in the land areas and at least partially in contact with the materials in the junction areas. This imparts an essentially three-dimensional structure to the fibrous layer of thermoplastic adhesive material, which in itself is essentially a two-dimensional structure of relatively small thickness, as compared to the dimension in length and width directions. Thereby, the fibrous thermoplastic adhesive material may provide cavities to cover the absorbent material in the land area, and thereby immobilizes this absorbent material, which may be 100% or less of SAP.

In one embodiment, the absorbent structure may have an intake factor greater than 3 according to U.S. Pat. No. 7,073,373, wherein the intake factor is defined as the absorbent core permeability divided by the normalized retention capacity (which is defined by the Retention Capacity Test—also according to U.S. Pat. No. 7,073, 373).

In one embodiment, the absorbent composite has a body fluid absorption greater than 75 g/100 cm², according to U.S. Pat. No. 6,649,810.

In one embodiment, a target location of the absorbent article may have a wicking value greater than 36%, according to U.S. Pat. No. 6,383,960.

In one embodiment, the absorbent article may have a bending stiffness between 0.05-1.0 gf, according to U.S. Pat. No. 5,810,796.

In one embodiment, the absorbent article may have a crotch fluid absorption rate greater than 3 g/sec according to U.S. Pat. No. 6,649,810. In one embodiment, a freeze-dried composite of the absorbent composite may have an intake rate of at least about 1.9 cubic centimeters (cc) of liquid/second at 80% composite saturation according to U.S. Pat. No. 6,689,934.

### Core Wraps

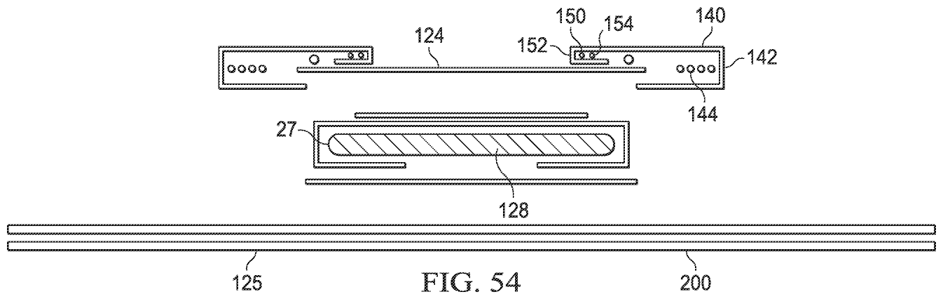

The core wrap may be made of a single substrate, material, or nonwoven folded around the absorbent material, or may comprise two (or more) substrates, materials, or nonwovens which are attached to another. Typical attachments are the so-called C-wrap and/or sandwich wrap. In a C-wrap, the longitudinal and/or transversal edges of one of the substrates are folded over the other substrate to form flaps. These flaps are then bonded to the external surface of the other substrate, typically by gluing. Other techniques may be used to form a core wrap. For example, the longitudinal and/or transversal edges of the substrates may be bonded together and then folded underneath the absorbent core 128 and bonded in that position one embodiment of a core wrap is illustrated in FIG. 54.

The core wrap may be at least partially sealed adjacent all of the sides of the absorbent core 128 so that substantially no absorbent material leaks out of the core. By “substantially no absorbent material” it is meant that less than 5%, less than 2%, less than 1%, or about 0% by weight of absorbent material escape the core wrap. The term “seal” is to be understood in a broad sense. The seal does not need to be continuous along the whole periphery of the core wrap but may be discontinuous along part or the whole of it, such as formed by a series of seal points spaced on a line. A seal may be formed by gluing and/or thermal bonding. The core wrap may also be formed by a single substrate which may enclose as in a parcel wrap the absorbent material and be sealed along the front side and rear side of the core and one longitudinal seal. The core wrap may comprise one or more nonwoven layers formed of a co-form material having a pulp fiber to synthetic fiber ratio of from 1:3 to 3:1, i.e. 25%:75% pulp fiber:synthetic fiber to 75%:25% pulp fiber:synthetic fiber.

### Channels

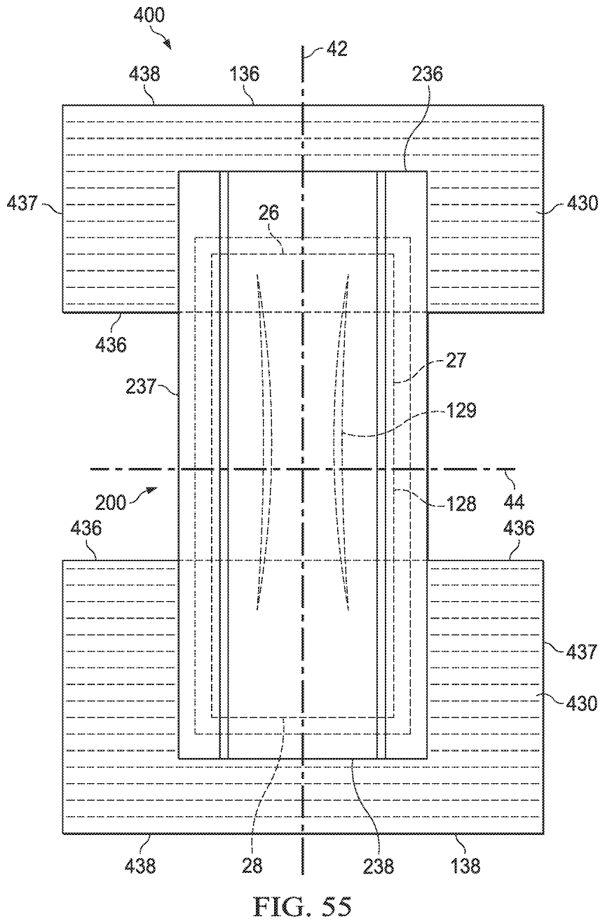

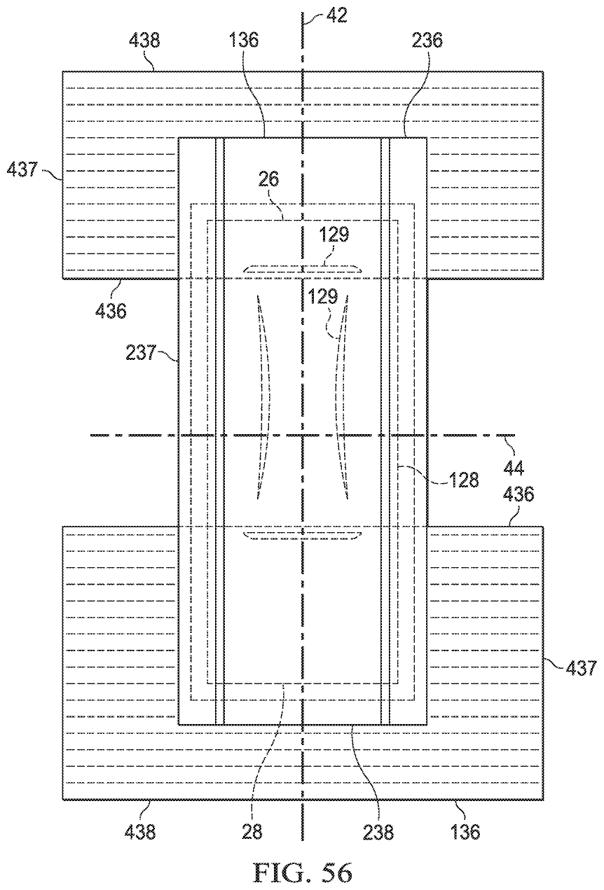

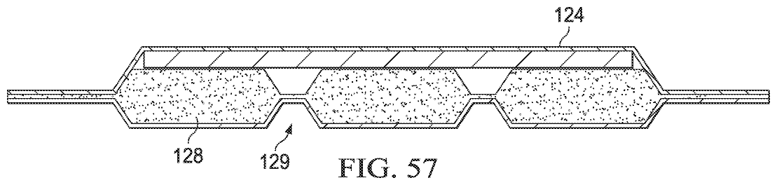

As illustrated in FIGS. 55-57, the absorbent core 128 may comprise at least one channel 129, which is at least partially oriented in the longitudinal direction of the absorbent article 100 (i.e., has a longitudinal vector component). Other channels may be at least partially oriented in the lateral direction (i.e., has a lateral vector component) or in any other direction. In the following, the plural form “channels” will be used to mean “at least one channel”. The channels 129 may be circular, oblong, or be in the shape of a variety of other closed polygons. The channels 129 may be formed in various ways. For example, the channels 129 may be formed by zones within the absorbent core 128 and may be substantially free of, or free of, absorbent material, in particular, SAP. In addition or alternatively, the channels 129 may also be formed by continuously or discontinuously bonding the top side of the core wrap to the bottom side of the core wrap through the absorbent core in the channel area. The channels 129 may be continuous or intermittent. The liquid management system, which may comprise an acquisition layer, distribution layer or both, or another layer of the absorbent article 100, may also comprise channels 129, which may or not correspond to the channels 129 of the absorbent core, as described in more detail below.

The absorbent core 128 may comprise more than two channels, for example, at least 3, at least 4, etc. Shorter channels may also be present, for example in the rear waist region or the front waist region of the core as represented by the pair of channels 129 towards the front of the absorbent article 100. The channels 129 may comprise one or more pairs of channels symmetrically arranged, or otherwise arranged relative to the longitudinal axis 42 or the lateral axis 44.

At least some or all of the channels 129 may be permanent channels, meaning their integrity is at least partially maintained both in the dry state and in the wet state. Permanent channels may be obtained by provision of one or more adhesive materials, for example, the fibrous layer of adhesive material or construction glue that helps adhere a substrate with an absorbent material within the walls of the channel. Permanent channels may also be formed by bonding the upper side and lower side of the core wrap and/or the topsheet 124 to the backsheet 125 together through the channels. Typically, an adhesive may be used to bond both sides of the core wrap or the topsheet and the backsheet through the channels, but it is possible to bond via other known processes, such as pressure bonding, ultrasonic bonding, heat bonding, or combinations thereof. The core wrap or the topsheet 124 and the backsheet 125 may be continuously bonded or intermittently bonded along the channels 129. The channels 129 may advantageously remain or become visible at least through the topsheet 124 and/or backsheet 125 when the absorbent article 100 is fully loaded with a fluid. This may be obtained by making the channels substantially free of SAP, so they will not swell, and sufficiently large so that they will not close when wet. Furthermore, bonding the core wrap to itself or the topsheet 124 to the backsheet 125 in the channel area may be advantageous.

Absorbent cores and/or liquid management systems without any channels are also within the scope of the present disclosure. These cores may include airfelt-free cores, SAP/pulp cores, pulp cores, or other cores known to those of skill in the art.

### Liquid Management Systems (LMS)

One function of the LMS is to quickly acquire the fluid and/or partition and/or distribute it to the absorbent core 128 in an efficient manner. The LMS may comprise one or more layers, which may form a unitary layer or may remain as discrete layers which may be attached to each other. The LMS may comprise two layers: a distribution layer and an acquisition layer disposed between the absorbent core 128 and the topsheet 124, but the present disclosure is not limited to such a configuration.

The LMS may be substantially free (e.g., 80%, 85%, 90%, 95%, or 99% free of) or completely free of SAP. The LMS may also comprise one or more of a variety of other suitable types of materials, such as opened-cell foam, cellulosic fibers, chemically treated cellulosic fibers, air-laid fibers, wt-laid fibers or carded, resin bonded nonwoven materials, for example. Suitable example LMSs are described in WO 2000/59430 (Daley), WO 95/10996 (Richards), U.S. Pat. No. 5,700,254 (McDowall), and WO 02/067809 (Graef), for example.

### Distribution Layers

The LMS may comprise a distribution layer. The distribution layer may comprise at least 50% or more by weight of cross-linked cellulose fibers, for example. The cross-linked cellulosic fibers may be crimped, twisted, or curled, or a combination thereof including crimped, twisted, and curled. Alternatively the distribution layer may comprise, wet-laid structure, air-laid structures, open cell foams, and microfiber structures. This type of material is disclosed in U.S. Pat. Publ. No. 2008/0312622 A1 (Hundorf).

### Acquisition Layers

The LMS may alternatively or additionally comprise an acquisition layer. The acquisition layer may be disposed, for example, between the distribution layer and the topsheet 124. The acquisition layer may be or may comprise a non-woven material, such as an SMS or SMMS material, comprising a spunbonded, a melt-blown and a further spunbonded layer or alternatively a carded chemical-bonded nonwoven. The acquisition layer may comprise air or wet-laid cellulosic, cross-linked cellulosic, or synthetic fibers, or blends thereof. The acquisition layer may comprise a roll-stock web of synthetic fibers (which may be processed to increase void space, such as by solid state formation), or a combination of synthetic and cellulosic fibers, bonded together to form a highloft material. Alternatively, the acquisition layer may comprise absorbent open cell foam. The nonwoven material may be latex bonded.

### Packages

The absorbent articles of the present disclosure may be placed into packages. The packages may comprise polymeric films and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

Accordingly, packages of the absorbent articles of the present disclosure may have an In-Bag Stack Height of less than about 130 mm, less than about 115 mm, less than about 100 mm, less than about 85 mm, but greater than about 75 mm, according to the In-Bag Stack Height Test described herein. Alternatively, packages of the absorbent articles of the present disclosure may have an In-Bag Stack Height of from about 75 mm to about 130 mm, from about 75 mm to about 115 mm, from about 85 mm to about 100 mm, according to the In-Back Stack Height Test described herein.

What is claimed is:

1. A method of providing a plurality of disposable absorbent articles, the method comprising steps of:
providing a pant, comprising:
a first center chassis comprising a first topsheet, a first backsheet and a first absorbent core; the first topsheet forming a portion of the interior surface of the first pant; the first backsheet forming a portion of the exterior surface of the first pant; the first absorbent core is disposed between the first topsheet and the first backsheet; a first elastomeric belt flap comprising at least one of:
(a) a first graphic; and/or
(b) a colored nonwoven; and/or
(c) a tinted nonwoven;
a second elastomeric belt flap;
a first refastenable seam;
a second refastenable seam;
providing a taped diaper, comprising:
a second center chassis comprising a second topsheet, a second backsheet and a second absorbent core;
the second topsheet forming a portion of the interior surface of the second pant;
the second backsheet forming a portion of the exterior surface of the second pant;
the second absorbent core is disposed between the second topsheet and the second backsheet;
a first elastomeric side flap;
a second elastomeric side flap;
wherein the pant and the taped diaper:
(a) have the same brand; and/or
(b) have the same sub-brand; and/or
(c) are made by or for the same manufacturer; and/or
(d) have commonly assigned trademarks;
wherein the plurality of absorbent articles form an array at a common point of sale.

2. The method of claim 1, wherein a center chassis of one or more of the absorbent articles comprises at least one of:
(a) a first graphic; and/or
(b) a colored nonwoven; and/or
(c) a tinted nonwoven;

3. The method of claim 1, wherein at least one of the absorbent articles comprises a textured topsheet.

4. The method of claim 1, wherein at least one of the absorbent articles comprises a discrete waistband.

5. The method of claim 4, wherein the discrete waistband is disposed in one or both of a front waist region and a back waist region, and wherein the waistband is disposed on an interior surface of the at least one absorbent article.

6. The method of claim 1, wherein at least one of the absorbent articles comprise an outer leg cuff, and wherein at least a portion of the outer leg cuff is formed by a film, a nonwoven, and an elastic material disposed between the film and the nonwoven.

7. The method of claim 1, wherein at least one of the absorbent articles comprise an inner leg cuff and an outer leg cuff, and wherein the inner and outer leg cuffs are formed from a folded nonwoven web and comprise elastics disposed between layers of the folded nonwoven web.

8. The method of claim 1, wherein at least one of the absorbent articles comprise a portion of a sensor system.

9. The method of claim 1, wherein at least one of the absorbent articles comprise an absorbent core comprising channels disposed therein.

10. The method of claim 9, wherein the channels are at least substantially devoid of absorbent material.

11. The method of claim 1, wherein the first elastomeric belt flap extends outwardly from a first end edge of the first center chassis and a longitudinally opposing second elastomeric belt flap extends outwardly from a longitudinally opposing second end edge of the first center chassis, and wherein a longitudinally distal edge of the first elastomeric belt flap and a longitudinally distal edge of the longitudinally opposing second elastomeric belt flap form a portion of a waist opening.

12. The method of claim 1, wherein the first elastomeric side flap extends outwardly from a first side edge of the third center chassis and a laterally opposing second elastomeric side flap extends outwardly from a laterally opposing second side edge of the third center chassis, and wherein a longitudinally distal edge of the third center chassis and longitudinally distal edges of the laterally opposing first and second elastomeric side flaps form a portion of a waist opening.

13. The method of claim 1, wherein at least one of the absorbent articles comprise an apertured elastomeric film.

14. The method of claim 1, wherein at least one of the absorbent articles comprise an elastic strand material.

15. The method of claim 1, wherein one or both of the elastomeric belt flaps are non-rectangular.

16. The method of claim 1, wherein the elastomeric side flaps are non-rectangular.

17. The method of claim 1, wherein one or both of the elastomeric belt flaps comprise a nonwoven layer having a basis weight between 6 and 36 g/m2.

18. The method of claim 1, wherein the elastomeric side flaps comprise a nonwoven layer having a basis weight between 6 and 36 g/m2.

19. The method of claim 1, wherein one or both of the elastomeric belt flaps comprise at least two visually distinct textured zones.

20. The method of claim 1, wherein the elastomeric side flaps comprise at least two visually distinct textured zones.

21. The method of claim 1, wherein one or both of the first elastomeric belt flap and second elastomeric belt flap comprise a graphic and wherein the graphic disposed on one or both of the belt flaps are substantially aligned with a graphic disposed on the center chassis.

22. The method of claim 1, wherein the first side flap comprises a first graphic and the second side flap comprises a second graphic and wherein the first graphic and the second graphic are substantially aligned with a graphic disposed on the center chassis.

23. The method of claim 1, wherein one or more of the absorbent articles comprises a fastening system comprising one or more of a fastener tab, primary fastener and mating fastener

24. The method of claim 1, wherein the pant and the taped diaper are arranged on the same shelf display.

25. The method of claim 1, wherein each of the articles comprise a package and at least two of the packages comprise substantially overlapping weight ranges.
